(19) United States
(12) Patent Application Publication
(10) Pub. No.: US 2023/0381522 A1
(43) Pub. Date: Nov. 30, 2023

(54) LOW ENERGY MULTIMODAL STIMULATION

(71) Applicant: Medtronic, Inc., Minneapolis, MN (US)

(72) Inventors: Andrew J. Cleland, St. Paul, MN (US); Brooke G. Kelley, Brooklyn Center, MN (US); Juan G. Hincapie, Maple Grove, MN (US); Vinod Sharma, Maple Grove, MN (US); Jeffery M. Kramer, St. Louis Park, MN (US)

(21) Appl. No.: 18/248,219

(22) PCT Filed: Oct. 8, 2021
(86) PCT No.: PCT/US2021/054306

§ 371 Date: Apr. 6, 2023

Related U.S. Application Data
(60) Provisional application No. 63/089,536, filed on Oct. 8, 2020.
(60) Provisional application No. 63/253,469, filed on Oct. 7, 2021.

(51) Int. Cl. A61N 1/36

(52) U.S. Cl. CPC A61N 1/36178; A61N 1/36071; A61N 1/36171

(57) ABSTRACT

This disclosure is directed to devices, systems, and techniques for delivering various stimulation patterns. In some examples, a method includes generating, by stimulation generation circuitry, a first train of electrical stimulation pulses at a first frequency to a first target tissue, and generating, by the stimulation generation circuitry, a second train of electrical stimulation pulses at a second frequency to a second target tissue different from the first target tissue, wherein at least some electrical stimulation pulses of the first train of electrical stimulation pulses are interleaved with at least some electrical stimulation pulses of the second train of electrical stimulation pulses, and wherein the first frequency is greater than the second frequency.

This application claims priority to U.S. Provisional Patent Application No. 63/253,469, filed on Oct. 7, 2021, and U.S. Provisional Patent Application No. 63/089,536, filed on Oct. 8, 2020, the entire contents of which are incorporated herein by reference.

### TECHNICAL FIELD

This disclosure generally relates to medical devices, and more specifically, electrical stimulation.

### BACKGROUND

Medical devices may be external or implanted and may be used to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS), respectively.

### SUMMARY

In general, the disclosure is directed to devices, systems, and techniques for providing therapy to a patient (e.g., pain relief therapy) by using multimodal stimulation having a low energy. In other words, the multimodal stimulation may be delivered using fewer pulses and/or pulses requiring less energy than other multimodal stimulation that may provide pain relief or other therapy to the patient. For example, the multimodal stimulation may include delivering first stimulation at a first frequency to a first target tissue and delivering second stimulation at a second frequency to a second target tissue different from the first target tissue. The first stimulation and the second stimulation may be interleaved over time such that one or more pulses from the first stimulation alternate with one or more pulses from the second stimulation. The first stimulation may include one, two, three, or more different interleaved pulse trains having the same or different individual frequencies. In this manner, the first stimulation may have an average frequency determined by the collective individual frequencies of the different interleaved pulse trains, wherein the average frequency is higher than any of the individual frequencies of the pulses of respective pulse trains. In some examples, the interpulse frequency may change from pulse to pulse within the first stimulation. These techniques may require less energy over time than the delivery of uniform or higher frequency pulses while achieving efficacious therapy for the patient.

In one example, the disclosure describes a method that includes generating, by stimulation generation circuitry, a first train of electrical stimulation pulses at a first frequency to a first target tissue; and generating, by the stimulation generation circuitry, a second train of electrical stimulation pulses at a second frequency to a second target tissue different from the first target tissue, wherein at least some electrical stimulation pulses of the first train of electrical stimulation pulses are interleaved with at least some electrical stimulation pulses of the second train of electrical stimulation pulses, and wherein the first frequency is greater than the second frequency.

In another example, the disclosure describes a system including stimulation generation circuitry configured to generate and deliver electrical stimulation therapy; and processing circuitry configured to control the stimulation generation circuitry to: generate a first train of electrical stimulation pulses at a first frequency to a first target tissue; and generate a second train of electrical stimulation pulses at a second frequency to a second target tissue different from the first target tissue, wherein at least some electrical stimulation pulses of the first train of electrical stimulation pulses are interleaved with at least some electrical stimulation pulses of the second train of electrical stimulation pulses, and wherein the first frequency is greater than the second frequency.

In another example, the disclosure describes a non-transitory computer-readable medium including instructions that, when executed, cause processing circuitry to control stimulation generation circuitry to: generate a first train of electrical stimulation pulses at a first frequency to a first target tissue; and generate a second train of electrical stimulation pulses at a second frequency to a second target tissue different from the first target tissue, wherein at least some electrical stimulation pulses of the first train of electrical stimulation pulses are interleaved with at least some electrical stimulation pulses of the second train of electrical stimulation pulses, and wherein the first frequency is greater than the second frequency.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system that includes an implantable medical device (IMD) configured to deliver spinal cord stimulation (SCS) therapy and an external programmer, in accordance with one or more techniques of this disclosure.

FIG. 2 is a block diagram illustrating an example configuration of components of an IMD, in accordance with one or more techniques of this disclosure.

FIG. 3 is a block diagram illustrating an example configuration of components of an external programmer, in accordance with one or more techniques of this disclosure.

FIG. 4 is a timing diagram illustrating examples of electrical stimulation pulses delivered according to different stimulation patterns.

FIG. 5 is a timing diagram illustrating examples of electrical stimulation pulses delivered according to different stimulation patterns.

FIG. 6 is a timing diagram illustrating examples of electrical stimulation pulses delivered according to different stimulation patterns.

FIG. 7 is a flow diagram illustrating an example technique for delivering electrical stimulation according to a specific pattern of pulses having different pulse frequencies.

FIG. 8 is a flow diagram illustrating an example technique for adjusting the frequency of prime stimulation pulses within range of frequencies.

FIG. 9 is a flow diagram illustrating an example technique for reducing stimulation intensity of first stimulation and/or second stimulation over time while maintaining effective therapy.

FIG. 10 is a flow diagram illustrating an example technique for adjusting a parameter value that defines prime stimulation pulses based on an evoked compound action potential (ECAP) elicited by a base stimulation pulse.

Like reference characters denote like elements throughout the description and figures.

### DETAILED DESCRIPTION

The disclosure describes examples of medical devices, systems, and techniques for providing therapy to a patient (e.g., pain relief therapy) by using multimodal stimulation having a low energy. The oscillatory electromagnetic fields applied to neural structures induce changes in synaptic plasticity upon modulation of two different cell populations: Neurons and glial cells. This is concurrent with the effects on neurons such as action potential generation or blockade by the stimulation of mechanosensitive fibers to mask (or close the gate to) nociceptive signals travelling to the brain. In addition, glial cells are immunocompetent cells that constitute the most common cell population in the nervous system and play a fundamental role in the development and maintenance of chronic neuropathic pain. Glial cells are responsible for monitoring the status of the nervous system by using constant chemical communication with neurons and other glial cells. Microglia are the glial cells in charge of monitoring the brain and spinal cord. Following a nerve (or brain) injury, these cells become activated and respond to any stimulus that is considered a threat to Central Nervous System (CNS) homeostasis. This activation involves morphological changes in the microglia accompanied by changes in chemotaxis and phagocytic activity, as well as the release of chemokines and cytokines that induce a response from the immune system. It has been shown that microglia are the CNS immediate responders to injury. Injury also triggers the activation of astrocytes, glial cells that monitor the synaptic clefts and thus are involved in synaptic plasticity via the regulation of neuro and glial transmitter molecules and involvement of immune cells for synaptic pruning. Astrocyte activation and regulation is sustained for longer time and thus it can be hypothesized that astrocytes play an important role in changes affecting synaptic plasticity in chronic pain. There is experimental evidence that supports this hypothesis. It is worth noting that at the Peripheral Nervous System (PNS), oligodendrocytes, Schwann cells and satellite glial cells, similar to astroglia, play similar roles. Calcium ions and phosphorylating processes mediated by ATP play an important role in glial response to injury. Electrical impulses induce changes in the concentration of calcium ions in the astrocytes, which propagates between astrocytes via calcium waves. This, in turn, signals the release of transmitters such as glutamate, adenosine and ATP, even after sodium channel blockade, which modulates both neuronal excitability and synaptic transmission. The presence of an external oscillatory electrical field then provides a stimulus for glial cells to affect synapses that have been negatively affected by injury. The electrical field provides a priming response that moves the function of the synapse towards a normal state.

Without being bound by theory, it is possible to electrically stimulate glial cells as their response (glial depolarization, release/uptake of ions, release of glial transmitters) depends on the specific parameters such as amplitude, frequency, phase polarity, waveform shape, and width (in the case of rectangular waveforms) of the stimulation. For example, the release of glutamate from astrocytes may be modulated in proportion to the amount of anodic current administered during biphasic pulsed stimulation. Monophasic cathodic stimulation of hippocampal astrocytes promotes the release of glutamate. The introduction of an anodic component decreases the amount of glutamate released. Given that the glial cells and neurons respond differently to electrical fields; it is then possible to differentially modulate the response of these cell populations with distinctly different electrical parameters. This theory sets a mechanistic basis of multimodal stimulation. Subthreshold stimulation with an electromagnetic field set at an optimum frequency, amplitude, waveform, width and phase may modulate the behavior of glial cells and the way they interact with neurons at the synaptic level. Thus, multimodal modulation provides the ability to control the balance of glutamate and glutamine in a calcium dependent manner and the possibility of modulating such balance in the appropriate manner with electromagnetic fields.

Electromagnetic fields modulate the expression of genes and proteins, which are involved in many processes involving synaptic plasticity, neuroprotection, neurogenesis, and inflammation. A genome-wide expression analysis of ipsilateral DC and DRG tissues obtained from an animal model of chronic neuropathic pain, in which SCS was applied continuously for 72 hours, provided findings that informed development of the multimodal methodologies described below. Without wishing to be bound by theory, the gene expression results indicated that the analgesic effect was likely induced at the molecular level in addition to, or independently of, the electric field blocking or masking nerve signaling. For example, SCS was identified to have upregulated genes for calcium binding proteins (Cabp), cytokines (Tnf, 116, 111 b, Cxcl16, lfg), cell adhesion (ltgb) and specific immune response proteins (Cd68, Tlr2), all of which have been linked to glial activation. Modulation parameters, particularly the oscillation frequency and amplitude, may play an important role in the mode of action.

According to one exemplary aspect of the disclosure, a method for multimodal modulation utilizes a composite electric field with at least one component oscillating at a frequency higher than the other component. This composite electric field is believed to provide pain relief that exceeds the amount of pain relief provided by either electric field on its own. The electrical field of the higher frequency “priming” component provides a persistent electrochemical potential that may facilitate the stimulation of nerves by another component that is oscillating at a lower frequency. Without being bound by theory, the priming component can lower the threshold for depolarization of nerve fibers while simultaneously modulating glial activation. The priming component may also lower the impedance of the stimulated tissue, which allows for better penetration of the electric field into the neural tissue. The frequent pulsing of the priming component also contributes to a lower threshold for depolarization of nerve fibers via membrane integration of the electrical stimulus. Additionally, the priming component may contribute to neuronal desynchronization, which is a mechanism that helps with the reestablishment of neuronal circuits that have been unnaturally synchronized to maintain a nociceptive input into the brain.

In the disclosed prime multimodal modulation technique, a mechanism of depolarization is combined with amplitudes lower or slightly higher than the Paresthesia Threshold (PT), so the patient may or may not experience tingling even though tonic stimulation is being applied. In certain embodiments, the composite signal, including the primary component that provides electrical stimulation at higher than the tonic frequencies, may activate the molecular mechanisms that allow for resetting of the synaptic plasticity to a state closer to the one previous to central sensitization induced by injury, thus providing a mechanism for long lasting pain relief

In certain embodiments, the Priming Frequency (PF) may be set to any frequency between 100 Hz to 600 kHz. When a charged-balanced pulsed rectangular electrical component, e.g., biphasic symmetric, biphasic asymmetric, capacitor coupled monophasic, is used, the Pulse Width (PW) of the priming component may be set as low as 10 μs and as large as allowed by the priming frequency. In some examples, the PW of pulses may be between approximately 150 to 300 μs, although other examples may have smaller or larger pulse widths. Either a voltage or current controlled composite signal may be used, although a current controlled signal may be more desirable as such signal does not depend on temporal impedance variations in the tissue being stimulated.

In certain embodiments, a first or priming frequency is between 50 Hz and 400 Hz (burst), or between 150 Hz and 300 Hz (average). According to embodiments, multiple signals can be multiplexed within a repeating set of N pulse spaces. Each pulse space within the pattern can correspond to a different electrical signal with respective parameters. The lower average frequency can be generated by multiplexing a second, tonic signal component in one of the N pulses. According to embodiments, the burst frequency of the priming frequency signal component can be an integer multiple (M) of the tonic signal frequency such that the tonic pulse space only includes a pulse every M times the N set of pulse spaces are repeated. The blank pulse space results in a burst of N−1 pulses at the “burst” frequency, followed by a “missed” pulse resulting in a lower “average” frequency over the set of N pulses. As used herein, the average frequency of the priming signal is calculated separate without including pulses associated with the tonic signal. In some embodiments, the priming signal can be delivered to a different physical location using a different set of electrodes relative to the tonic signal. In another exemplary embodiment, the first or priming frequency is set to 400 Hz (burst), or 200 Hz (average). In certain embodiments, each pulse within a burst may be provided on a separate program for different groups of electrodes, with a configuration set to allow for individual amplitude variability.

In further exemplary embodiments, a second or tonic component is set at a frequency of about 50 Hz, interleaved into the treatment to account for the average priming frequency, though other tonic values and ranges are contemplated herein, e.g., 20 Hz to 200 Hz, 20 Hz to 100 Hz, 30 Hz to 80 Hz, etc.

Disclosed herein are apparatus and methods for managing pain in a patient by using multimodal stimulation of neural structures, with an electromagnetic signal having multiple components of characteristic frequencies, amplitudes, and phase polarities. Multimodal modulation for pain management, in accordance with the disclosure, contemplates the use of oscillating electromagnetic fields which is applied via an array of electrodes (referred as contacts or leads) to a particular neural structure using temporal and amplitude characteristics, to modulate glial and neuronal interactions as the mechanism for relieving chronic pain. More specifically, exemplary aspects provide an apparatus and method for modulating the expression of genes involved in diverse pathways including inflammatory/immune system mediators, ion channels and neurotransmitters, in both the Spinal Cord (SC) and Dorsal Root Ganglion (DRG). In one exemplary embodiment, such expression modulation is caused by spinal cord stimulation or peripheral nerve stimulation. In one embodiment, the amplitudes and frequencies of the signal or signals used to create the multimodal stimulation of neural structures may be optimized for pain relief and low power usage in an implantable multimodal signal generator, as described herein.

According to one exemplary embodiment, apparatuses and methods provide for managing pain in a patient by using multiplexed stimulation signals to target different neural structures such that the multiple stimulation signals are multiplexed in the time domain, hereafter referred to as “differential target multiplexed stimulation.” For instance, a signal generator can multiplex signals that can have different signal characteristics (e.g., pulse frequency, amplitude, or pulse duration) to generate differential target multiplexed stimulation for pain management. In accordance with aspects of the disclosure, the output of the signal generator can be used to produce separate oscillating electromagnetic fields (stimulation signals, such as pulses or continuous signals) which can be applied to different set of a plurality of electrodes (also referred as contacts). The electrodes can be part of a lead that is designed to apply the respective stimulation signals to different parts of a particular neural structure.

Various aspects of the disclosure relate to the use of a variety of temporal and amplitude characteristics in order to modulate glial and neuronal interactions as the mechanism for relieving chronic pain. The multiplexed stimulation signals have characteristics that allow for a synergistic targeting of glial cells and neurons in a differential manner. For instance, disclosed are embodiments relating to an apparatus and method for modulating the expression of genes and proteins involved in diverse pathways, including inflammatory/immune system mediators, ion channels and neurotransmitters, associated with the interaction of glia and neurons in neural tissue. In embodiments, such expression modulation may be caused by any of spinal cord stimulation, dorsal root ganglion stimulation, brain stimulation, or peripheral nerve stimulation. In some embodiments, the amplitudes, phase polarity, waveforms, and frequencies of the signals combined to create the differential target multiplexed stimulation of neural structures may be optimized for pain relief and low power usage in an implantable signal generator, as described herein.

In embodiments of differential target multiplexed stimulation therapy, a set of high frequency charge-balanced biphasic pulsed signals in which the polarity of the first phase of the high frequency signals may be either cathodic or anodic is utilized. In embodiments, a set of low frequency signals is used that may have waveform characteristics different from those of the high frequency signals. The polarity of the first phase of the biphasic charge-balanced low frequency signals may be either cathodic or anodic. The high and low frequency stimulation signals can be delivered to the neural tissues by multiplexing individual pulses from each via respective sets of electrodes. In certain embodiments, the respective sets of electrodes can be co-located in close proximity to the same neural tissue (e.g., near the same vertebrae).

Although electrical stimulation is generally described herein in the form of electrical stimulation pulses, electrical stimulation may be delivered in non-pulse form in other examples. For example, electrical stimulation may be delivered as a signal having various waveform shapes, frequencies, and amplitudes. Therefore, electrical stimulation in the form of a non-pulse signal may be a continuous signal than may have a sinusoidal waveform or other continuous waveform.

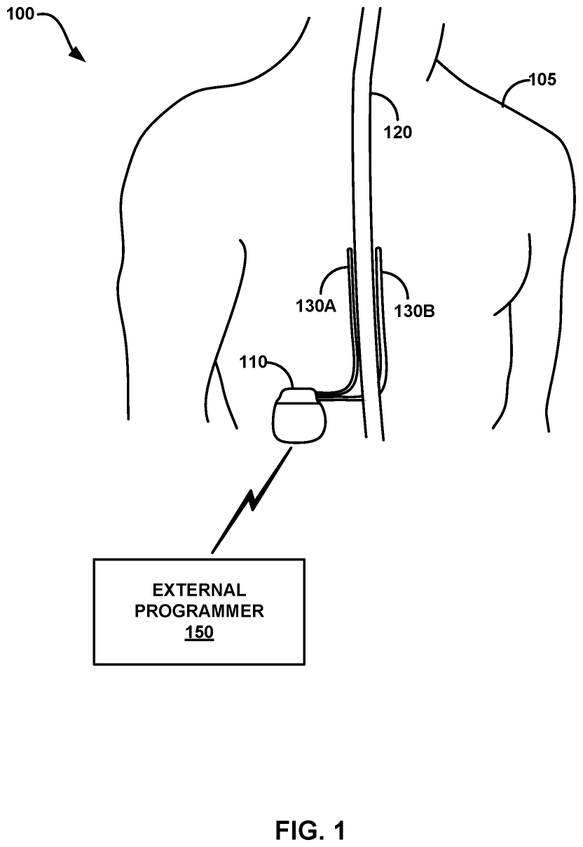

FIG. 1 is a conceptual diagram illustrating an example system 100 that includes an implantable medical device (IMD) 110 configured to deliver spinal cord stimulation (SCS) therapy, processing circuitry 140, and an external programmer 150, in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices.

As shown in FIG. 1, system 100 includes an IMD 110, leads 130A and 130B, and external programmer 150 shown in conjunction with a patient 105, who is ordinarily a human patient. In the example of FIG. 1, IMD 110 is an implantable electrical stimulator that is configured to generate and deliver electrical stimulation therapy to patient 105 via one or more electrodes of electrodes of leads 130A and/or 130B (collectively, “leads 130”), e.g., for relief of chronic pain or other symptoms. In other examples, IMD 110 may be coupled to a single lead carrying multiple electrodes or more than two leads each carrying multiple electrodes. IMD 110 may be a chronic electrical stimulator that remains implanted within patient 105 for weeks, months, or even years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to percutaneously implanted leads. In some examples, IMD 110 uses one or more leads, while in other examples, IMD 110 is leadless.

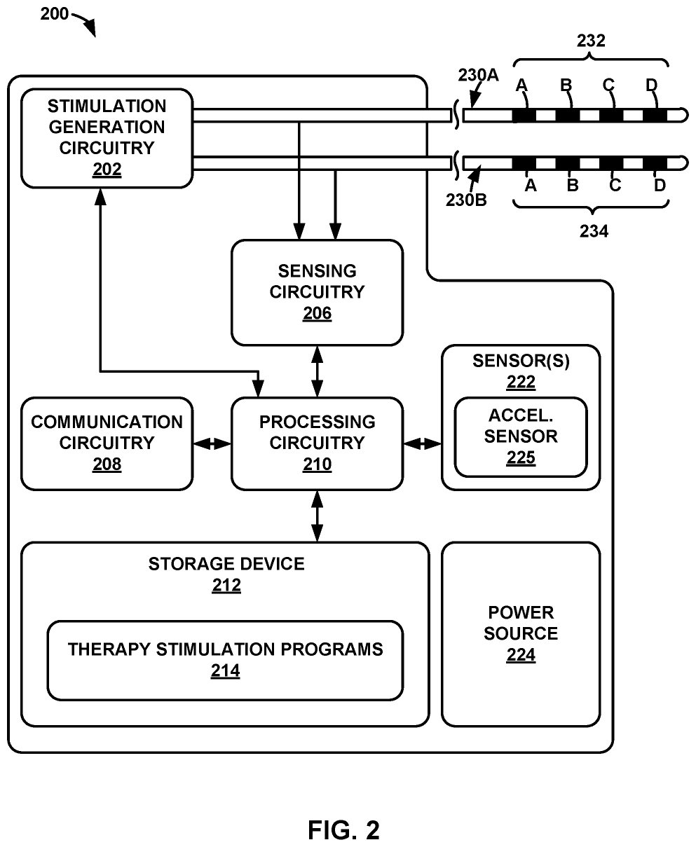

IMD 110 may be constructed of any polymer, metal, or composite material sufficient to house the components of IMD 110 (e.g., components illustrated in FIG. 2) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks. In other examples, IMD 110 may be implanted within other suitable sites within patient 105, which may depend, for example, on the target site within patient 105 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge the rechargeable power source.

Electrical stimulation energy, which may be constant current or constant voltage-based pulses, for example, is delivered from IMD 110 to one or more target tissue sites of patient 105 via one or more electrodes (not shown) of implantable leads 130. In the example of FIG. 1, leads 130 carry electrodes that are placed adjacent to the target tissue of spinal cord 120. One or more of the electrodes may be disposed at a distal tip of a lead 130 and/or at other positions at intermediate points along the lead. Leads 130 may be implanted and coupled to IMD 110. The electrodes may transfer electrical stimulation generated by an electrical stimulation generator in IMD 110 to tissue of patient 105. Although leads 130 may each be a single lead, lead 130 may include a lead extension or other segments that may aid in implantation or positioning of lead 130. In some other examples, IMD 110 may be a leadless stimulator with one or more arrays of electrodes arranged on a housing of the stimulator rather than leads that extend from the housing. In addition, in some other examples, system 100 may include one lead or more than two leads, each coupled to IMD 110 and directed to similar or different target tissue sites.

The electrodes of leads 130 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration.

The deployment of electrodes via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 130 are linear leads having 8 ring electrodes along the axial length of the lead. In another example, the electrodes are segmented rings arranged in a linear fashion along the axial length of the lead and at the periphery of the lead.

The stimulation parameter of a therapy stimulation program that defines the stimulation pulses of electrical stimulation therapy by IMD 110 through the electrodes of leads 130 may include information identifying which electrodes have been selected for delivery of stimulation according to a stimulation program, the polarities of the selected electrodes, i.e., the electrode combination for the program, and voltage or current amplitude, pulse frequency, pulse width, pulse shape of stimulation delivered by the electrodes. These stimulation parameters of stimulation pulses are typically predetermined parameter values determined prior to delivery of the stimulation pulses (e.g., set according to a stimulation program). However, in some examples, system 100 changes one or more parameter values automatically based on one or more factors or based on user input.

Although FIG. 1 is directed to SCS therapy, e.g., used to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 100 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 105.

In some examples, lead 130 includes one or more sensors configured to allow IMD 110 to monitor one or more parameters of patient 105, such as patient activity, pressure, temperature, or other characteristics. The one or more sensors may be provided in addition to, or in place of, therapy delivery by lead 130.

IMB 110 is configured to deliver electrical stimulation therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation, which may be in the form of electrical stimulation pulses or continuous waveforms. In some examples, the target tissue includes nerves, smooth muscle or skeletal muscle. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical or lumbar regions. Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may be reduce the perception of pain by patient 105, and thus, provide efficacious therapy results. For example, as described herein, electrical stimulation may be directed to glial cells while other electrical stimulation (delivered by different electrode combination) is directed to neurons.

IMD 110 generates and delivers electrical stimulation therapy to a target stimulation site within patient 105 via the electrodes of leads 130 to patient 105 according to one or more therapy stimulation programs. A therapy stimulation program defines values for one or more parameters that define an aspect of the therapy delivered by IMD 110 according to that program. For example, a therapy stimulation program that controls delivery of stimulation by IMD 110 in the form of pulses may define values for voltage or current pulse amplitude, pulse width, and pulse rate (e.g., pulse frequency) for stimulation pulses delivered by IMD 110 according to that program.

A user, such as a clinician or patient 105, may interact with a user interface of an external programmer 150 to program IMD 110. Programming of IMD 110 may refer generally to the generation and transfer of commands, programs, or other information to control the operation of IMD 110. In this manner, IMD 110 may receive the transferred commands and programs from external programmer 150 to control electrical stimulation therapy. For example, external programmer 150 may transmit therapy stimulation programs, stimulation parameter adjustments, therapy stimulation program selections, user input, or other information to control the operation of IMD 110, e.g., by wireless telemetry or wired connection. As described herein, stimulation delivered to the patient may include control pulses, and, in some examples, stimulation may include control pulses and informed pulses.

In some cases, external programmer 150 may be characterized as a physician or clinician programmer if it is primarily intended for use by a physician or clinician. In other cases, external programmer 150 may be characterized as a patient programmer if it is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change electrical stimulation therapy. In general, a physician or clinician programmer may support selection and generation of programs by a clinician for use by IMD 110, whereas a patient programmer may support adjustment and selection of such programs by a patient during ordinary use. In other examples, external programmer 150 may include, or be part of, an external charging device that recharges a power source of IMD 110. In this manner, a user may program and charge IMD 110 using one device, or multiple devices.

As described herein, information may be transmitted between external programmer 150 and IMD 110. Therefore, IMD 110 and external programmer 150 may communicate via wireless communication using any techniques known in the art. Examples of communication techniques may include, for example, radiofrequency (RF) telemetry and inductive coupling, but other techniques are also contemplated. In some examples, external programmer 150 includes a communication head that may be placed proximate to the patient's body near the IMD 110 implant site to improve the quality or security of communication between IMD 110 and external programmer 150. Communication between external programmer 150 and IMD 110 may occur during power transmission or separate from power transmission.

In some examples, IMD 110, in response to commands from external programmer 150, delivers electrical stimulation therapy according to a plurality of therapy stimulation programs to a target tissue site of the spinal cord 120 of patient 105 via electrodes (not depicted) on leads 130. In some examples, IMD 110 modifies therapy stimulation programs as therapy needs of patient 105 evolve over time. For example, the modification of the therapy stimulation programs may cause the adjustment of at least one parameter of the plurality of informed pulses. When patient 105 receives the same therapy for an extended period, the efficacy of the therapy may be reduced. In some cases, parameters of the plurality of informed pulses may be automatically updated.

In the case of multimodal modulation of the spinal cord, various multi-contact leads can be positioned in the epidural space to stimulate the cell populations already described. In one particular arrangement, the leads can be positioned parallel to each other, although not necessarily coplanar within the epidural space. Two eight-contact electrode arrays can be used for the disclosed multimodal modulation techniques. Note that the polarity of the leads can also be customized during the programming stage, either as bipolar, monopolar, or guarded cathode configurations. Another example of a possible electrode array arrangement includes leads arranged staggered relative to each other. The customization and optimization of therapy may comprise the positioning of the leads within the epidural space at appropriate vertebral segments in either type of lead arrangement.

Other arrangements may be used to stimulate different places along the spinal canal, e.g., the leads do not need to be parallel. For example, in one arrangement, one lead can be dedicated to deliver a signal at the spinal cord at a given vertebral level, while the other provides a signal either more caudad or cephalad relative to the position of the other lead. Leads can be, in principle, located at any vertebral level in the spinal cord, or could also be positioned peripherally, because the principle behind multimodal modulation applies to peripheral glial cells that survey the axons.

Furthermore, the multimodal stimulation electromagnetic field's location and penetration may be also utilized for customization and optimization of therapy by delivering multimodal stimulation signals to particular arrays of electrodes within each lead by setting monopolar, bipolar, or guarded cathode arrangements of such electrode arrays. For example, therapy for a patient with low back pain that extends into one of the lower extremities may require positioning the stimulation leads in a staggered arrangement within the epidural space along vertebral levels thoracic 8 (TS) and thoracic 12 (T12). An array of electrodes in the more cephalad of the leads may be set to monopolar, bipolar or guarded cathode arrangement. Another array of electrodes in the more caudad of the leads may be set to monopolar, bipolar or guarded cathode arrangement. The clinician will be able to customize the electrode array setting in a methodical manner such that therapy can be optimized for based on feedback from the patient.

Optionally, pain relief may also be used by position the leads in the neighborhood of a peripheral nerve. Peripheral Nerve Stimulation (PNS) is an alternative therapy for chronic pain in which a target nerve has been identified to be the source of pain. The current understanding of the therapeutic effects of PNS is also based on the gate control theory. However, axons of sensory neurons in peripheral nerves are surrounded by glial cells that are known to respond accordingly to the frequency characteristics of a stimulus.

Multimodal peripheral nerve stimulation involves the positioning of one or more stimulation leads around or in the neighborhood of a target nerve. The leads are connected to a signal generator with multimodal capacity as described herein. Multimodal stimulation is delivered to the neural tissue consisting of neuron axons and their corresponding glial cells (Schwann cells) according to the principles and methods described in this application. The leads may implant to be positioned around the target nerve using an invasive surgical approach or percutaneously utilizing a needle cannula.

Alternatively, as would be the case for the stimulation of target nerves that are close to the skin surface (such as the vagus nerve, nerves in the joints of the extremities, etc.) the leads may be arranged inside a conductive biocompatible pad for delivery of the multimodal electromagnetic field transcutaneously. This embodiment constitutes Transcutaneous Electrical Nerve Multimodal Stimulation (TENMS). In this embodiment, the priming high frequency component of the multimodal signal lowers the impedance of the skin and subcutaneous tissue and allows for better penetration of the tonic signal. The priming signal also provides a modulating signal for perisynaptic glial cells in the neuromuscular junction. These cells are known to discriminate different stimulation patterns and respond accordingly, thus allowing for modulation of the synapse with multimodal stimulation. The tonic component of the multimodal signal is used to stimulate the neuronal axon at lower thresholds.

FIG. 2 is a block diagram illustrating an example configuration of components of IMD 200, in accordance with one or more techniques of this disclosure. IMD 200 may be an example of IMD 110 of FIG. 1. In the example shown in FIG. 2, IMD 200 includes stimulation generation circuitry 202, sensing circuitry 206, communication circuitry 208, processing circuitry 210, storage device 212, sensor(s) 222, and power source 224.

In the example shown in FIG. 2, storage device 212 stores therapy stimulation programs 214 within storage device 212. Each stored therapy stimulation program of therapy stimulation programs 214 defines values for a set of electrical stimulation parameters (e.g., a stimulation parameter set for each pulse train or each slot of a series of slots), such as a stimulation electrode combination, electrode polarity, current or voltage amplitude, pulse width, pulse rate, and pulse shape.

Accordingly, in some examples, stimulation generation circuitry 202 generates electrical stimulation signals in accordance with the electrical stimulation parameters noted above. Other ranges of stimulation parameter values may also be useful and may depend on the target stimulation site within patient 105. While stimulation pulses are described, stimulation signals may be of any form, such as continuous-time signals (e.g., sine waves) or the like. Stimulation generation circuitry 202 includes a plurality of pairs of voltage sources, current sources, voltage sinks, or current sinks connected to each of electrodes 232, 234 such that each pair of electrodes has a unique signal circuit. In other words, in these examples, each of electrodes 232, 234 is independently controlled via its own signal circuit (e.g., via a combination of a regulated voltage source and sink or regulated current source and sink), as opposed to switching signals between electrodes 232, 234.

In other examples, however, switch circuitry may include one or more switch arrays, one or more multiplexers, one or more switches (e.g., a switch matrix or other collection of switches), or other electrical circuitry configured to direct stimulation signals from stimulation generation circuitry 202 to one or more of electrodes 232, 234, or directed sensed signals from one or more of electrodes 232, 234 to sensing circuitry 206. Stimulation generation circuitry 202 and/or sensing circuitry 206 may include switch circuitry to direct signals to and/or from one or more of electrodes 232, 234, which may or may not also include a distinct switch circuitry.

Sensing circuitry 206 monitors signals from any combination of electrodes 232, 234. In some examples, sensing circuitry 206 includes one or more amplifiers, filters, and analog-to-digital converters. Sensing circuitry 206 may be used to sense physiological signals, such as evoked compound action potentials (ECAPs). In some examples, sensing circuitry 206 detects ECAPs from a particular combination of electrodes 232, 234. In some cases, the particular combination of electrodes for sensing ECAPs includes different electrodes than a set of electrodes 232, 234 used to deliver stimulation pulses. Alternatively, in other cases, the particular combination of electrodes used for sensing ECAPs includes at least one of the same electrodes as a set of electrodes used to deliver stimulation pulses to patient 105. Sensing circuitry 206 may provide signals to an analog-to-digital converter, for conversion into a digital signal for processing, analysis, storage, or output by processing circuitry 210.

Communication circuitry 208 supports wireless communication between IMD 200 and an external programmer (not shown in FIG. 2) or another computing device under the control of processing circuitry 210. Processing circuitry 210 of IMD 200 may receive, as updates to programs, values for various stimulation parameters such as amplitude and electrode combination, from the external programmer via communication circuitry 208. Updates to the therapy stimulation programs 214 may be stored within storage device 212. Communication circuitry 208 in IMD 200, as well as telemetry circuits in other devices and systems described herein, such as the external programmer, may accomplish communication by radiofrequency (RF) communication techniques. In addition, communication circuitry 208 may communicate with an external medical device programmer (not shown in FIG. 2) via proximal inductive interaction of IMD 200 with the external programmer. The external programmer may be one example of external programmer 150 of FIG. 1. Accordingly, communication circuitry 208 may send information to the external programmer on a continuous basis, at periodic intervals, or upon request from IMD 110 or the external programmer.

Processing circuitry 210 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 controls stimulation generation circuitry 202 to generate stimulation signals according to therapy stimulation programs 214 and ECAP test stimulation programs 216 stored in storage device 212 to apply stimulation parameter values specified by one or more of programs, such as amplitude, pulse width, pulse rate, and pulse shape of each of the stimulation signals.

In the example shown in FIG. 2, the set of electrodes 232 includes electrodes 232A, 232B, 232C, and 232D, and the set of electrodes 234 includes electrodes 234A, 234B, 234C, and 234D. In other examples, a single lead may include all eight electrodes 232 and 234 along a single axial length of the lead. Processing circuitry 210 also controls stimulation generation circuitry 202 to generate and apply the stimulation signals to selected combinations of electrodes 232, 234. In some examples, stimulation generation circuitry 202 includes a switch circuit (instead of, or in addition to, separate switch circuitry) that may couple stimulation signals to selected conductors within leads 230, which, in turn, deliver the stimulation signals across selected electrodes 232, 234. Such a switch circuit may be a switch array, switch matrix, multiplexer, or any other type of switching circuit configured to selectively couple stimulation energy to selected electrodes 232, 234 and to selectively sense bioelectrical neural signals of a spinal cord of the patient (not shown in FIG. 2) with selected electrodes 232, 234.

Electrodes 232, 234 on respective leads 230 may be constructed of a variety of different designs. For example, one or both of leads 230 may include one or more electrodes at each longitudinal location along the length of the lead, such as one electrode at different perimeter locations around the perimeter of the lead at each of the locations A, B, C, and D. In one example, the electrodes may be electrically coupled to stimulation generation circuitry 202, e.g., via separate switch circuitry and/or switching circuitry of the stimulation generation circuitry 202, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 230. These and other constructions may be used to create a lead with a complex electrode geometry.

Although sensing circuitry 206 is incorporated into a common housing with stimulation generation circuitry 202 and processing circuitry 210 in FIG. 2, in other examples, sensing circuitry 206 may be in a separate housing from IMD 200 and may communicate with processing circuitry 210 via wired or wireless communication techniques.

Storage device 212 may be configured to store information within IMD 200 during operation. Storage device 212 may include a computer-readable storage medium or computer-readable storage device. Storage device 212 may include, for example, random access memories (RAM), dynamic random access memories (DRAM), static random access memories (SRAM), ferroelectric random access memories (FRAM), magnetic discs, optical discs, flash memories, or forms of electrically programmable memories (EPROM) or electrically erasable and programmable memories (EEPROM). In some examples, storage device 212 is used to store data indicative of instructions for execution by processing circuitry 210. As discussed above, storage device 212 is configured to store therapy stimulation programs 214.

Sensor(s) 222 may include one or more sensing elements that sense values of a respective patient parameter. As described, electrodes 232 and 234 may be the electrodes that sense the characteristic value of the ECAP. Sensor(s) 222 may include one or more accelerometers, optical sensors, chemical sensors, temperature sensors, pressure sensors, or any other types of sensors. Sensor(s) 222 may output patient parameter values that may be used as feedback to control delivery of therapy. IMD 200 may include additional sensors within the housing of IMD 200 and/or coupled via one of leads 130 or other leads. In addition, IMD 200 may receive sensor signals wirelessly from remote sensors via communication circuitry 208, for example. In some examples, one or more of these remote sensors may be external to patient (e.g., carried on the external surface of the skin, attached to clothing, or otherwise positioned external to patient 105).

Power source 224 is configured to deliver operating power to the components of IMD 200. Power source 224 may include a battery and a power generation circuit to produce the operating power. In some examples, the battery is rechargeable to allow extended operation. In some examples, recharging is accomplished through proximal inductive interaction between an external charger and an inductive charging coil within IMD 200. In other examples, power source 224 may include one or more primary batteries that are not rechargeable. Power source 224 may include any one or more of a plurality of different battery types, such as nickel cadmium batteries and lithium ion batteries.

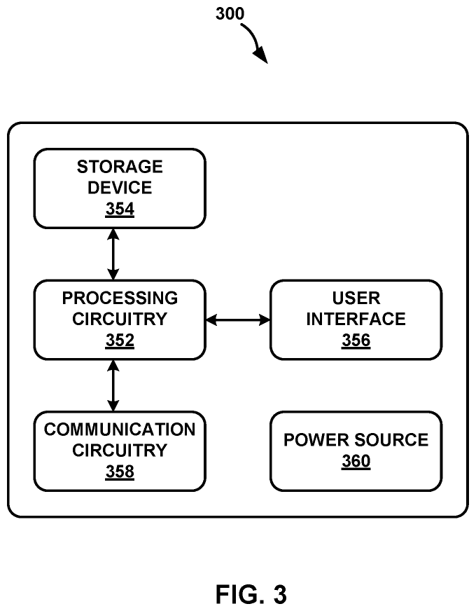

FIG. 3 is a block diagram illustrating an example configuration of components of external programmer 300, in accordance with one or more techniques of this disclosure. External programmer 300 may be an example of external programmer 150 of FIG. 1. Although external programmer 300 may generally be described as a hand-held device, external programmer 300 may be a larger portable device or a more stationary device. In addition, in other examples, external programmer 300 may be included as part of an external charging device or include the functionality of an external charging device. As illustrated in FIG. 3, external programmer 300 may include processing circuitry 352, storage device 354, user interface 356, communication circuitry 358, and power source 360. Storage device 354 may store instructions that, when executed by processing circuitry 352, cause processing circuitry 352 and external programmer 300 to provide the functionality ascribed to external programmer 300 throughout this disclosure. Each of these components, circuitry, or modules, may include electrical circuitry that is configured to perform some, or all of the functionality described herein. For example, processing circuitry 352 may include processing circuitry configured to perform the processes discussed with respect to processing circuitry 352.

In general, external programmer 300 includes any suitable arrangement of hardware, alone or in combination with software and/or firmware, to perform the techniques attributed to external programmer 300, and processing circuitry 352, user interface 356, and communication circuitry 358 of external programmer 300. In various examples, external programmer 300 may include one or more processors, such as one or more microprocessors, DSPs, ASICs, FPGAs, or any other equivalent integrated or discrete logic circuitry, as well as any combinations of such components. External programmer 300 also, in various examples, may include a storage device 354, such as RAM, ROM, PROM, EPROM, EEPROM, flash memory, a hard disk, a CD-ROM, including executable instructions for causing the one or more processors to perform the actions attributed to them. Moreover, although processing circuitry 352 and communication circuitry 358 are described as separate modules, in some examples, processing circuitry 352 and communication circuitry 358 are functionally integrated. In some examples, processing circuitry 352 and communication circuitry 358 correspond to individual hardware units, such as ASICs, DSPs, FPGAs, or other hardware units.

Storage device 354 (e.g., a storage device) may store instructions that, when executed by processing circuitry 352, cause processing circuitry 352 and external programmer 300 to provide the functionality ascribed to external programmer 300 throughout this disclosure. For example, storage device 354 may include instructions that cause processing circuitry 352 to obtain a parameter set from memory, select a spatial electrode movement pattern, or receive a user input and send a corresponding command to IMD 200, or instructions for any other functionality. In addition, storage device 354 may include a plurality of programs, where each program includes a parameter set that defines stimulation pulses, such as control pulses and/or informed pulses. Storage device 354 may also store data received from a medical device (e.g., IMD 110). For example, storage device 354 may store ECAP related data recorded at a sensing module of the medical device, and storage device 354 may also store data from one or more sensors of the medical device. This ECAP related data may include ECAP information transmitted from an implantable medical device, such as IMD 110.

User interface 356 may include a button or keypad, lights, a speaker for voice commands, a display, such as a liquid crystal (LCD), light-emitting diode (LED), or organic light-emitting diode (OLED). In some examples the display includes a touch screen. User interface 356 may be configured to display any information related to the delivery of electrical stimulation, identified patient behaviors, sensed patient parameter values, patient behavior criteria, or any other such information. In addition, as described herein, processing circuitry 352 may control user interface 356 to present graphical representations of ECAP information transmitted by IMD 110. User interface 356 may also receive user input via user interface 356. The input may be, for example, in the form of pressing a button on a keypad or selecting an icon from a touch screen. The input may request starting or stopping electrical stimulation, the input may request a new spatial electrode movement pattern or a change to an existing spatial electrode movement pattern, of the input may request some other change to the delivery of electrical stimulation.

Communication circuitry 358 may support wireless communication between the medical device and external programmer 300 under the control of processing circuitry 352. Communication circuitry 358 may also be configured to communicate with another computing device via wireless communication techniques, or direct communication through a wired connection. In some examples, communication circuitry 358 provides wireless communication via an RF or proximal inductive medium. In some examples, communication circuitry 358 includes an antenna, which may take on a variety of forms, such as an internal or external antenna.

Examples of local wireless communication techniques that may be employed to facilitate communication between external programmer 300 and IMD 110 include RF communication according to the 802.11 or Bluetooth® specification sets or other standard or proprietary telemetry protocols. In this manner, other external devices may be capable of communicating with external programmer 300 without needing to establish a secure wireless connection. As described herein, communication circuitry 358 may be configured to transmit a spatial electrode movement pattern or other stimulation parameter values to IMD 110 for delivery of electrical stimulation therapy.

In some examples, selection of stimulation parameters or therapy stimulation programs are transmitted to the medical device for delivery to a patient (e.g., patient 105 of FIG. 1). In other examples, the therapy may include medication, activities, or other instructions that patient 105 must perform themselves or a caregiver perform for patient 105. In some examples, external programmer 300 provides visual, audible, and/or tactile notifications that indicate there are new instructions. External programmer 300 requires receiving user input acknowledging that the instructions have been completed in some examples.

Power source 360 is configured to deliver operating power to the components of external programmer 300. Power source 360 may include a battery and a power generation circuit to produce the operating power. In some examples, the battery is rechargeable to allow extended operation. Recharging may be accomplished by electrically coupling power source 360 to a cradle or plug that is connected to an alternating current (AC) outlet. In addition, recharging may be accomplished through proximal inductive interaction between an external charger and an inductive charging coil within external programmer 300. In other examples, traditional batteries (e.g., nickel cadmium or lithium ion batteries) may be used. In addition, external programmer 300 may be directly coupled to an alternating current outlet to operate.

The architecture of external programmer 300 illustrated in FIG. 3 is shown as an example. The techniques as set forth in this disclosure may be implemented in the example external programmer 300 of FIG. 3, as well as other types of systems not described specifically herein. Nothing in this disclosure should be construed so as to limit the techniques of this disclosure to the example architecture illustrated by FIG. 3.

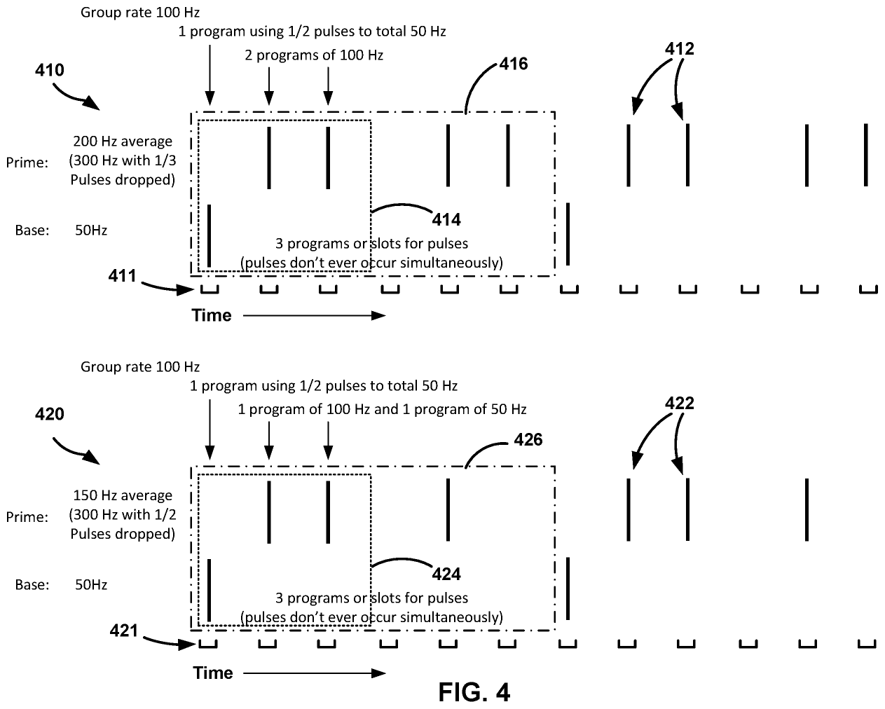

FIG. 4 includes timing diagrams illustrating examples of electrical stimulation pulses delivered according to different stimulation patterns. As shown in FIG. 4, timing diagrams 410 and 420 provide examples of different methods for delivering multimodal stimulation. In some examples, pulses of electrical stimulation can be delivered to two different target tissues, such as the prime pulses in a higher frequency train being delivered to glial cells, as one example, and base pulses of a lower frequency delivered to a different target tissue, such as neurons associated with the spinal cord. In some examples, each pulse of both trains of pulses are delivered in respective slots of a series of slots. In one example, there are four slots that represent a respective period of time during which a single stimulation pulse can be delivered. Put another way, 4 programs (or respective pulse trains) can be active at the same time, which one pulse from each program being deliverable in its respective slot. The series of slots then continues to repeat over time. In this manner, the pulses of the 4 programs (or respective pulse trains) are at least partially interleaved over time.

In one example not illustrated, the prime stimulation includes pulses delivered during the second, third, and fourth slot of each series of slots. The group rate determines the frequency that the series of slots is repeated. Therefore, if the group rate is 300 Hz, the three pulses in the second, third, and fourth slots have a maximum interpulse frequency of 1200 Hz and an average of 900 Hz because the first slot of every series of slots is occupied by the program of the base stimulation delivered to a different target tissue via a different electrode combination. In one example, the pulse of the base stimulation train of pulses is only delivered once every sixth occurrence of series of slots. When the pulse is not delivered in a series of slots, that slot remains empty such that no pulse is delivered. For a group rate of 300 Hz and a pulse delivered every sixth occurrence of the series of slots, the base pulse train would have a frequency of 50 Hz. Six repetitions of the four slots in each series of slots would be one complete repeating pattern for the prime and base pulse trains together. As IMD 200 continues to deliver pulses according to the programs and repeating series of slots, stimulation is delivered repeatedly with the pattern as long as stimulation is being delivered.

However, delivery of prime stimulation with pulse trains having average frequencies of 900 Hz may consume more power than may be necessary to treat the patient. Instead, the prime stimulation (e.g., the train of pulses delivered to glial cells) may be effective at much lower average pulse frequencies in other examples. A lower average frequency (e.g., about 100 Hz or greater or about 200 Hz or greater) is still greater than the base frequency of the lower pulse train, but it may enable IMD 200 to conserve power proportional the fewer number of pulses generated by IMD 200 than with typical prime pulse train average frequencies.

In the example of timing diagram 410, the first stimulation may include one or more trains of electrical stimulation pulses 412 in the upper train labeled as “prime” pulses. The second stimulation may include a train of electrical stimulation pulses 412 in the lower train labeled “base” pulses. Since series of slots 414 only includes three slots of slots 411, three pulses are shown in respective slots in time. Slots 411 illustrate each slot at a respective point in time at which IMD 200 can deliver a pulse.

The series of slots 414 from timing diagram 410 has a group rate of 100 Hz, and the second stimulation only includes a pulse in the first slot of every other series of pulses 414 such that the second stimulation has a frequency of 50 Hz. The first stimulation of the upper train includes two trains of pulses (but could include three or more distinct trains), where one train includes a pulse in the second slot of every series of pulses 414 and another train includes a pulse in the third slot of every series of pulses 414. In this manner, each train in the upper train has a respective frequency of 100 Hz, which results in an interpulse frequency of 300 Hz for the upper train and an average frequency of 200 Hz (e.g., an average frequency of 200 Hz over multiple series of pulses 414). Pattern 416 indicates one complete repeating pattern for the upper and lower trains together. As IMD 200 continues to deliver pulses according to the programs and repeating series of slots 414, stimulation is delivered repeatedly with pattern 416 as long as stimulation is being delivered. Generally, each frequency of the respective pulse trains in the upper train are greater than the frequency of the lower pulse train.

Timing diagram 420 is similar to timing diagram 410, but uses a different pattern of pulses 426 that reduces the average frequency of the prime pulses. In the example of timing diagram 420, the first stimulation may include one or more trains of electrical stimulation pulses 422 in the upper train labeled as “prime” pulses. The second stimulation may include a train of electrical stimulation pulses 422 in the lower train labeled “base” pulses. Since series of slots 424 only includes three slots of slots 421, three pulses are shown in respective slots in time. Slots 421 illustrate each slot at a respective point in time at which IMD 200 can deliver a pulse. Although the concept of slots 421 is used herein, IMD 200 may be configured to deliver pulses at any time based on other timing concepts that do not include slots, such as adjustable timing schedules or tracking individual pulse widths and interphase intervals, for example.

The series of slots 424 from timing diagram 420 has a group rate of 100 Hz, and the second stimulation only includes a pulse in the first slot of every other series of pulses 414 such that the second stimulation has a frequency of 50 Hz. The first stimulation of the upper train includes two trains of pulses (but could include three or more distinct trains), where one train includes a pulse in the second slot of every series of pulses 424 and another train includes a pulse in the third slot of every other series of pulses 424. In this manner, the train of second slot pulses in the upper train has a respective frequency of 100 Hz, and the train of third slot pulses in the upper train has a respective frequency of 200 Hz, which results in an interpulse frequency of 300 Hz for the upper train and an average frequency of 150 Hz (e.g., an average frequency of 150 Hz over multiple series of pulses 424). Pattern 426 indicates one complete repeating pattern for the upper and lower trains together. As IMD 200 continues to deliver pulses according to the programs and repeating series of slots 424, stimulation is delivered repeatedly with pattern 426 as long as stimulation is being delivered. Generally, each frequency of the respective pulse trains in the upper train (e.g., a 150 Hz average frequency) are greater than the frequency of the lower pulse train (e.g., 50 Hz).

The prime pulse frequencies and base pulse frequencies illustrated in the examples of timing diagrams 410 and 420 are merely some examples. On one example, the prime pulses may have an average frequency is selected from a frequency range from approximately 150 Hz to approximately 600 Hz. In other examples, the average frequency may be as low as 100 Hz or higher than 600 Hz. In other examples, the average frequency may be approximately 200 Hz or higher, and less than 600 Hz or less than 400 Hz. In some examples, IMD 200 may switch between programs in order to achieve other average frequencies for the prime and/or base stimulation pulses. For example, IMD 200 could switch between timing diagram 410 and 420 every certain number of series of pulses (e.g., every 2, 4, or 6 series of pulses) in order to achieve an average frequency for the prime pulses that falls between the average frequencies of each timing diagram. In one example, switching between the programs of timing diagrams 410 and 420 every two series of pulses would result in a prime average frequency of approximately 175 Hz over time.

Although the concept of a series of slots is provided as one example mechanism for managing the delivery of pulses for the first and second stimulation pulses, other management techniques may be used in other examples. For example, IMD 200 may have a flexible programming architecture that enables processing circuitry 210 to schedule different pulses for different electrode combinations at any frequency desired. For example, IMD 200 may simply run multiple different programs that define respective pulse trains interleaved as needed to achieve the respective frequencies of each pulse train.

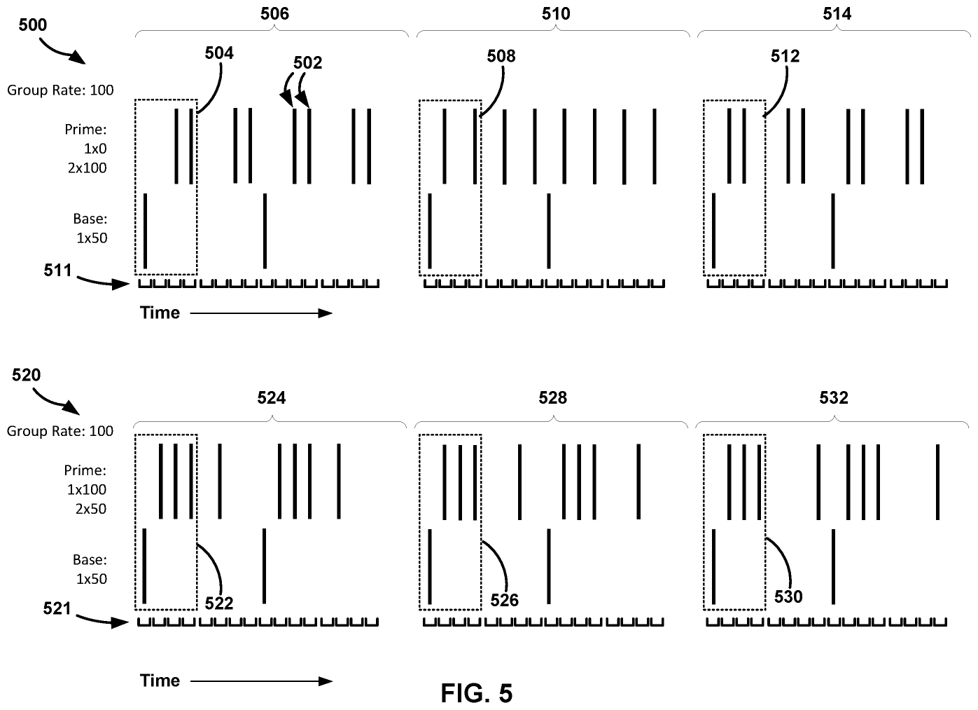

FIG. 5 includes timing diagrams 500 and 520 illustrating an example of electrical stimulation pulses delivered according to different stimulation patterns. Timing diagrams 500 and 520 may be similar to timing diagrams 410 and 420 of FIG. 4 because they are set to provide low energy stimulation as compared to higher frequency prime stimulation. As shown in timing diagram 500, different patterns 506, 510, and 514 are possible with different pulses 502 with a group rate of 100 Hz for series of slots that includes 4 slots. Slots 511 illustrate each slot at a respective point in time at which IMD 200 can deliver a pulse. In pattern 506, series of slots 504 has four slots where the first slot includes a pulses for the base stimulation to achieve 50 Hz stimulation, the second slot is left empty, and the third and fourth slots include respective 100 Hz pulse trains for the prime stimulation. Therefore, the resulting prime stimulation is delivered with an average of 200 Hz and an interpulse frequency of 400 Hz. In pattern 510, series of slots 508 has four slots where the first slot includes a pulses for the base stimulation to achieve Hz stimulation, the third slot is left empty, and the second and fourth slots include respective 100 Hz pulse trains for the prime stimulation. Therefore, the resulting prime stimulation is delivered with an average of 200 Hz and an interpulse frequency of 200 Hz. In pattern 514, series of slots 512 has four slots where the first slot includes a pulses for the base stimulation to achieve 50 Hz stimulation, the fourth slot is left empty, and the second and third slots include respective 100 Hz pulse trains for the prime stimulation. Therefore, the resulting prime stimulation is delivered with an average of 200 Hz and an interpulse frequency of 400 Hz. In some examples, IMD 200 may be configured to deliver stimulation using one of patterns 506, 510, or 514 on a repeating basis, or using a repeating combination of two or more of the patterns 506, 510, or 514 to achieve the average frequency of the prime pulses. In this manner, IMD 200 may utilize different series of slots (or changing programs) over time in order to achieve a desired average frequency of pulses for the prime pulses and/or the base pulses.

As shown in timing diagram 520, different patterns 524, 528, and 532 are possible with a group rate of 100 Hz for series of slots that includes 4 slots. Slots 521 illustrate each slot at a respective point in time at which IMD 200 can deliver a pulse. In pattern 524, series of slots 522 has four slots where the first slot includes a pulses for the base stimulation to achieve 50 Hz stimulation, the second slot includes pulses for a 100 Hz pulse train, and the third and fourth slots include pulses for respective 50 Hz pulse trains for the prime stimulation. Therefore, the resulting prime stimulation is delivered with an average of 200 Hz and an interpulse frequency of 400 Hz for three consecutive pulses. In pattern 528, series of slots 526 has four slots where the first slot includes a pulses for the base stimulation to achieve 50 Hz stimulation, the third slot includes pulses for a 100 Hz pulse train, and the second and fourth slots include pulses for respective 50 Hz pulse trains for the prime stimulation. Therefore, the resulting prime stimulation is delivered with an average of 200 Hz and an interpulse frequency of 400 Hz for three consecutive pulses. In pattern 532, series of slots 530 has four slots where the first slot includes a pulses for the base stimulation to achieve 50 Hz stimulation, the fourth slot includes pulses for a 100 Hz pulse train, and the second and third slots include pulses for respective 50 Hz pulse trains for the prime stimulation. Therefore, the resulting prime stimulation is delivered with an average of 200 Hz and an interpulse frequency of 400 Hz for three consecutive pulses. Although a group rate of 100 is described, the group rate may be adjusted according to the number of slots in the series of slots and the desired frequencies to achieve for each type of stimulation.

In some examples, it may be desirable for the pulses in the prime stimulation pulse train to be less than uniform, approaching random, or completely random. For this reason, pattern 532 may be used to achieve a more random pattern of pulses. In other examples, IMD 200 may alternative between two or more of the patterns of FIG. 5, or other patterns, in order to further change the pattern that is repeated during stimulation delivery for the prime stimulation.

In some examples, IMD 200 may be configured to deliver stimulation using one of patterns 524, 528, or 532 on a repeating basis, or using a repeating combination of two or more of the patterns 524, 528, or 532 to achieve the average frequency of the prime pulses. In this manner, IMD 200 may utilize different series of slots (or changing programs) over time in order to achieve a desired average frequency of pulses for the prime pulses and/or the base pulses.

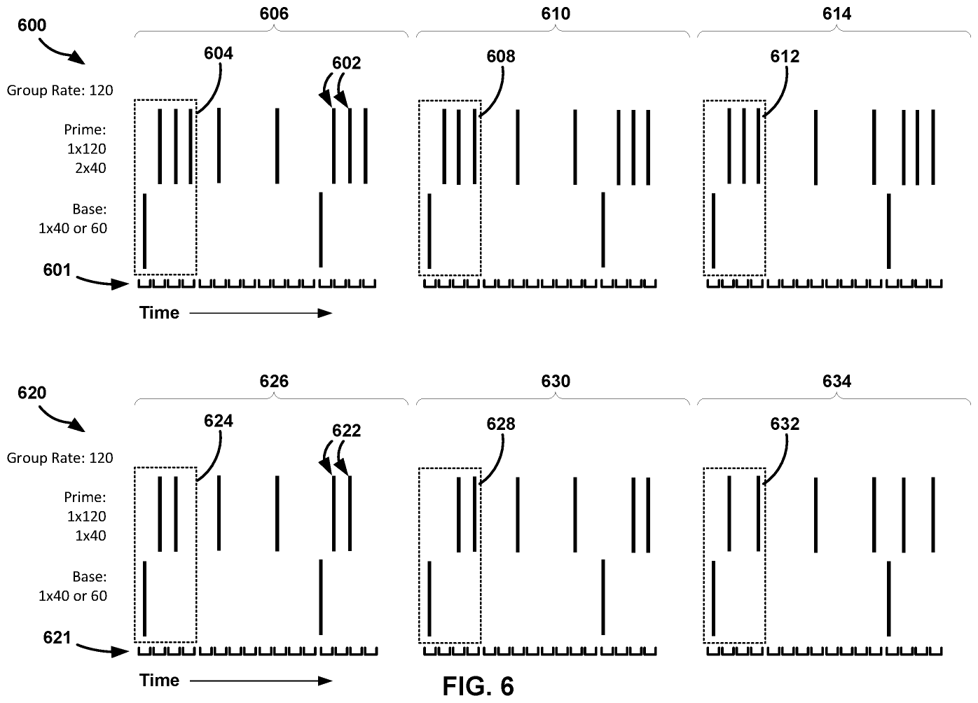

FIG. 6 is a timing diagram illustrating an example of electrical stimulation pulses delivered according to different stimulation patterns. Timing diagrams 600 and 620 may be similar to timing diagrams of FIGS. 4 and 5 because they are set to provide low energy stimulation as compared to higher frequency prime stimulation. However, timing diagrams 600 and 620 have a group rate of 120 Hz, which is slightly higher than the group rates of the series of slots of FIG. 5. As shown in the example of timing diagram 600, different patterns 606, 610, and 614 are possible with a group rate of 120 Hz for series of slots that includes 4 slots within which pulses 602 can be delivered. Slots 601 illustrate each slot at a respective point in time at which IMD 200 can deliver a pulse. In pattern 606, series of slots 604 has four slots where the first slot includes a pulses for the base stimulation to achieve 40 Hz stimulation, the second slot includes pulses for a 120 Hz pulse train, and the third and fourth slots include pulses for respective 40 Hz pulse trains for the prime stimulation. Therefore, the resulting prime stimulation is delivered with an average of 240 Hz and an interpulse frequency of 480 Hz for three consecutive pulses. In pattern 610, series of slots 608 has four slots where the first slot includes a pulses for the base stimulation to achieve 40 Hz stimulation, the third slot includes pulses for a 120 Hz pulse train, and the second and fourth slots include pulses for respective 40 Hz pulse trains for the prime stimulation. Therefore, the resulting prime stimulation is delivered with an average of 240 Hz and an interpulse frequency of 480 Hz for three consecutive pulses. In pattern 614, series of slots 612 has four slots where the first slot includes a pulses for the base stimulation to achieve 40 Hz stimulation, the fourth slot includes pulses for a 120 Hz pulse train, and the second and third slots include pulses for respective 40 Hz pulse trains for the prime stimulation. Therefore, the resulting prime stimulation is delivered with an average of 240 Hz and an interpulse frequency of 480 Hz for three consecutive pulses. Although a group rate of 120 is described, the group rate may be adjusted according to the number of slots in the series of slots and the desired frequencies to achieve for each type of stimulation. In other examples, the base stimulation may have a frequency of approximately 60 Hz. For any of the examples of herein, IMD 200 may switch the prime stimulation from one target tissue to another target tissue in order to achieve efficacious therapy.

In some examples, IMD 200 may be configured to deliver stimulation using one of patterns 606, 610, and 614 on a repeating basis, or using a repeating combination of two or more of the patterns 606, 610, and 614 to achieve the average frequency of the prime pulses. In this manner, IMD 200 may utilize different series of slots (or changing programs) over time in order to achieve a desired average frequency of pulses for the prime pulses and/or the base pulses.

As shown in the example of timing diagram 620, different patterns 626, 630, and 634 are possible with a group rate of 120 Hz for series of slots that includes 4 slots within which pulses 622 can be delivered. Slots 621 illustrate each slot at a respective point in time at which IMD 200 can deliver a pulse. In pattern 626, series of slots 624 has four slots where the first slot includes a pulse for the base stimulation to achieve 40 Hz stimulation, the second slot includes pulses for a 120 Hz pulse train, and the third slot includes pulses for a 40 Hz pulse train for the prime stimulation. No pulses are delivered in the fourth slot of series of slots 624 for the duration of pattern 626. Therefore, the resulting prime stimulation is delivered with an average of 180 Hz and an interpulse frequency of 480 Hz for two consecutive pulses in adjacent slots of slots 621. In pattern 630, series of slots 628 has four slots where the first slot includes a pulses for the base stimulation to achieve 40 Hz stimulation, the third slot includes pulses for a 120 Hz pulse train, and the fourth slot includes pulses for respective 40 Hz pulse trains for the prime stimulation. No pulses are delivered in the second slot of series of slots 628 for the duration of pattern 630. Therefore, the resulting prime stimulation is delivered with an average of 180 Hz and an interpulse frequency of 480 Hz for two consecutive pulses in adjacent slots 621. In pattern 634, series of slots 632 has four slots where the first slot includes a pulses for the base stimulation to achieve 40 Hz stimulation, the fourth slot includes pulses for a 120 Hz pulse train, and the second slot includes pulses for respective 40 Hz pulse trains for the prime stimulation. No pulses are delivered in the third slot of series of slots 632 for the duration of pattern 634. Therefore, the resulting prime stimulation is delivered with an average of 180 Hz and an interpulse frequency of 240 Hz for two consecutive prime pulses because prime pulses are not delivered in adjacent slots 621. Although a group rate of 120 is described, the group rate may be adjusted according to the number of slots in the series of slots and the desired frequencies to achieve for each type of stimulation. In other examples, the base stimulation may have a frequency of approximately 60 Hz. For any of the examples of herein, IMD 200 may switch the prime stimulation from one target tissue to another target tissue in order to achieve efficacious therapy.

In some examples, IMD 200 may be configured to deliver stimulation using one of patterns 626, 630, and 634 on a repeating basis, or using a repeating combination of two or more of the patterns 626, 630, and 634 to achieve the average frequency of the prime pulses. In this manner, IMD 200 may utilize different series of slots (or changing programs) over time in order to achieve a desired average frequency of pulses for the prime pulses and/or the base pulses.

In some examples, IMD 200 may change the order of pulses of one train of electrical stimulation pulses in the prime train with pulses of another train of electrical stimulation pulses over time to adjust a pulse pattern created by interleaving the at least of the electrical stimulation pulses of the trains of electrical stimulation pulses used to generate the overall prime train of stimulation pulses. In some examples, IMD 200 may alternate between two or more different patterns of pulses, where the different patterns include different numbers of pulses resulting in different average frequencies for each pattern. Therefore, when delivered on an alternating basis, the resulting average frequency of the stimulation that includes the different patterns of pulses may be between the average frequencies of the low frequency patterns and the high frequency patterns. For example, stimulation that alternates between a pattern that includes a prime average frequency of 240 Hz and another pattern that includes a prime average frequency of 180 Hz would have an overall average pulse frequency of approximately 210 Hz.

Although the concept of slots is described for the purposes of the examples in FIGS. 4, 5, and 6 and elsewhere herein, IMD 200 may be configured to deliver pulses at any time based on other timing concepts that do not include slots, such as adjustable timing schedules or tracking individual pulse widths and interphase intervals to schedule different pulse trains without overlapping, for example. In some examples, if IMD 200 is not to overlap pulses from any trains, IMD 200 may move one or more pulses slightly in time (e.g., within a predetermined window from the scheduled time) in order to continue delivering each pulse train. If the pulses need to be moved outside of the predetermined window from the scheduled time, or if IMD 200 has instructions not to move pulses, IMD 200 may drop one of the overlapping pulses from one or more trains to prevent the overlap. IMD 200 may determine which pulse to drop based on one or more factors, such as which type of pulse is overlapping (e.g., IMD 200 may drop a prime pulse instead of a base pulse or a base pulse instead of a prime pulse), the frequencies of each pulse train (e.g., IMD 200 may drop a pulse from the higher frequency pulse train), the number of pulses in each train, whether or not the pulses are part of a train that is supra-perception threshold (e.g., the patient would notice a pulse being dropped), etc. In other examples, if the overlapping pulses are delivered by different electrode combinations that do not include any common electrodes (e.g., prime pulses via one electrode combination and base pulses via a different electrode combination), IMD 200 may continue to deliver the pulses even if overlapping if IMD 200 can deliver multiple pulses concurrently.

If programmer 300 determines that selected pulse frequency and pulse width combinations would result in overlapping pulses, programmer 300 may present a warning to the user indicating that one or more pulses may be dropped or moved due to a conflict with the pulse frequencies of the pulse trains. Programmer 300 may present alternative pulse frequency and/or pulse width options to avoid the conflict or present a confirmation button that the user can select to proceed with the selected stimulation despite the conflict. In other examples, programmer 300 may only present pulse frequency and pulse width options for user selection that will not result in any overlapping pulses. For example, after the user selects a pulse width for the different pulse trains, programmer 300 may present the available pulse frequency options that would not result in any overlapping pulses. In other examples, programmer 300 may enable the user to select the pulse frequencies first and then present available pulse widths that do not result in overlapping pulses.

In some examples, IMD 200 may select different prime pulse frequencies or prime pulse patterns, but a certain timing relationship between a base pulse and an adjacent prime pulse (e.g., before and/or after in time). For example, the overall stimulation efficacy of the base and prime pulses may be at least partially based on the timing between one or more prime pulses and an adjacent base pulse. This may be due to synaptic relationships between the two pulse types. In this example, IMD 200 may schedule prime pulses such that a certain interpulse interval is present between adjacent base and prime pulses. This interpulse interval may be selected in the range of 200 microseconds to 50 milliseconds. In some examples, the interpulse interval may be based on the pulse width of the base and prime pulses such that the start of the base pulse to the start of the prime pulse achieves a predetermined frequency, such as a frequency between approximately 100 Hz to 600 Hz. In some examples, the frequency may be approximately 200 Hz or 200 Hz averaged over time. In other examples, IMD 200 may vary the interpulse interval between the base pulse and adjacent prime pulses.

In general, a single IMD 200 may generate and deliver the first and second stimulation pulses (e.g., the prime and base pulses). In other examples, one IMDs may deliver the prime pulses to one anatomical region and a different and separate IMD may deliver the base pulses to a different anatomical region. Each of these different IMDs may communicate with each other to synchronize the delivery of the prime and base pulses. For example, the different IMDs may coordinate delivery to avoid overlap of the prime and base pulses or, alternatively, deliver base pulses at least partially at the same time as at least some of the prime pulses.

In some examples, the average frequency of the prime stimulation is selected from a frequency range from approximately 100 Hz to approximately 600 Hz. In another example, the average frequency of the prime stimulation is selected from a frequency range from approximately 150 Hz to approximately 300 Hz. In another example, the average frequency of the prime stimulation is selected from a frequency range from approximately 150 Hz to approximately 250 Hz. In another example, the average frequency of the prime stimulation is selected from a frequency range from approximately 180 Hz to approximately 240 Hz. In another example, the average frequency of the prime stimulation is selected from a frequency range from approximately 200 Hz to approximately 300 Hz. In another example, the average frequency of the prime stimulation is approximately 200 Hz. The frequency of the base stimulation may be selected from a frequency range from approximately 40 Hz to approximately 60 Hz. In some examples, IMD 200 may increase the amplitude of base stimulation until the patient achieves effective pain relief.

In some examples, IMD 200 may cycle between a first mode of a first period of time and a second mode of a second period of time, wherein the first mode comprises generating the first train of electrical stimulation pulses (e.g., the prime stimulation) at least partially interleaved with the second train of electrical stimulation pulses (e.g., the base stimulation). The second mode may include withholding generation of the first train of electrical stimulation pulses and the second train of electrical stimulation pulses. In some examples, the ratio of the first period to the second period of time is between approximately 1:1 and 1:3. In other examples, the ratio may be lower to enable much longer off periods for stimulation. In one example, the first period of time for stimulation is selected from a range from approximately 1 minute to approximately 30 minutes. In another example, the first period of time for stimulation is selected from a range from approximately 5 minute to approximately 15 minutes. In some examples, the on period for stimulation may be less than 1 minute or greater than 30 minutes. In other examples, the on period may be as short as 15 seconds or even less.

In some examples, the system may achieve low energy multimodal stimulation by cycling prime pulses (e.g., first stimulation) that have an inter-pulse frequency greater than 600 Hz. For example, IMD 200 may be configured to deliver prime pulses with an average frequency of approximately 900 Hz (which may have inter-pulse frequencies above and below 900 Hz, such as some inter-pulse frequencies as high as 1200 Hz or higher and some inter-pulse frequencies of 600 Hz or lower). Other example average frequencies above 600 Hz may include an average of 720 Hz, 800 Hz, 1000 Hz, etc. By cycling this prime pulse train with an average frequency above 600 Hz on and off over time according to any cycling schedule described herein, IMD 200 may effectively reduce the power required to deliver prime pulses while maintaining efficacy for the patient. In one example, IMD 200 may deliver the prime pulses with an average pulse frequency above 600 Hz for 5 minutes and then turn off, or withhold, the prime pulses for a duration of 15 minutes. Other example cycling times and ratios are described herein.

In some examples, the amplitude of pulses of the first train of electrical stimulation pulses (e.g., the prime stimulation) is below at least one of a perception threshold or a sensory threshold of a patient. In some examples, the amplitude of pulses of the second train of electrical stimulation pulses (e.g., the base stimulation) is below at least one of a perception threshold or a sensory threshold of a patient. In some examples, the system may automatically determine the perception or sensory threshold based on the intensity of pulses that elicits detectable ECAP signals.

The amplitude of a priming component may be set at a value below a Priming Perception Threshold (PPT), although setting it at or above the PPT is not excluded. The PPT may be found by slowly increasing the amplitude while feedback is obtained from the subject. Once the onset of perception is recorded, then the amplitude of the priming component may be changed to a value which is a percentage of the PPT (% PPT). With an exemplary PF of 200 Hz, the signal may be then set for a given time, e.g., 10-30 minutes, before an electric component set at a tonic frequency lower than the PF, e.g., 10 Hz to 199 kHz, is applied independently to other electrodes in the lead. In the prime mode of stimulation, the tonic frequency will be lower than the priming frequency but is not necessarily limited to a particular range of frequencies below the priming frequency.

The Pulse Width (PW) of a charge-balanced, e.g., a biphasic symmetric, biphasic asymmetric, or capacitor coupled monophasic, pulsed signal can be as low as 10 μs and as large as allowed by the set tonic frequency. In exemplary embodiments, the pulse width may be between about 100 and 500 microseconds, between about 100 and 400 microseconds, between about 150 and 200 microseconds, or any different value, range or combinations of pulse widths. In one example, the PW may be approximately 170 microseconds for prime stimulation pulses and 200 microseconds for base stimulation pulses. The PW of pulses of prime stimulation may be the same or different than the PW of base stimulation. The PW value may be tied to frequency in some examples. For example, lower frequency pulses may be capable of having longer pulse widths. In some examples, the system may maintain a certain charge density for prime and/or base pulses such that a change to pulse frequency may be accompanied by the system changing the pulse width in order to maintain the charge density of the stimulation.

The signal generation and delivery circuitry may also allow for modifying the duty cycles of pulsed width signals and various schemes in which the time of initial priming can be varied, as well as the times in which the priming signal is on or off relative to the time when the tonic signal is delivered. The amplitude of the tonic electrical component, which could be either voltage or current controlled, may be set above, below or at the Tonic Perception Threshold (TPT). PT may be obtained by increasing the amplitude of the tonic component while getting feedback from the patient. The tonic amplitude may then be set to a value corresponding to a percentage of the TPT (% TPT). In the prime multimodal modulation methods described herein both the priming component and the tonic component may be below 100 kHz, in one embodiment. In another embodiment, the tonic signal may be below 500 Hz. In still another embodiment, the tonic signal may be below 100 Hz. In one embodiment, the ratio of priming component frequency to tonic component frequency may be in the range of 2:1 to 40:1, 4:1 to 40:1, 10:1 to 40:1, 20:1 to 40:1, up to 70:1, up to 140:1, etc. depending on the specific values of the frequencies chosen.

In yet another embodiment of multimodal modulation therapy, the priming component may be biphasic in which the polarity of the first phase of the biphasic prime component may be either cathodic or anodic. With this embodiment, the tonic component may have characteristics that are different from those of the priming component. The tonic component may be biphasic with the polarity of the first phase of the biphasic tonic signal being either cathodic or anodic.

In exemplary embodiments of multimodal modulation therapy, an active recharge mode provides a recovery pulse that applies an equal charge in a direction opposite to the input, thus driving the waveform each way.

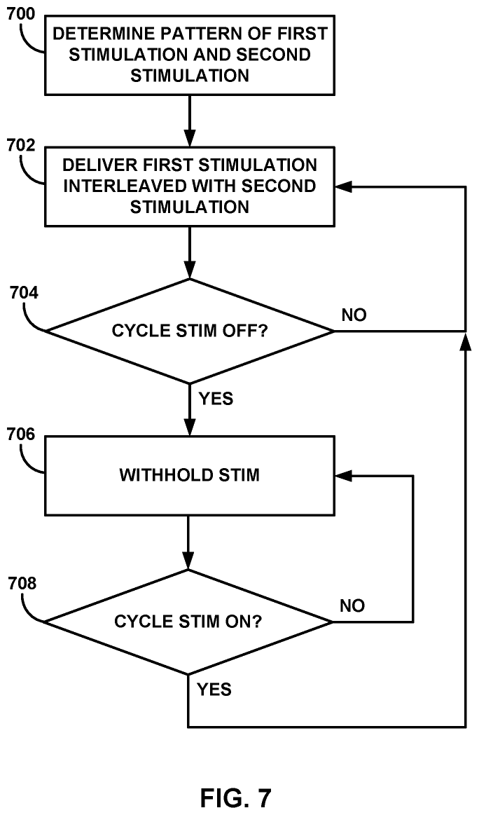

FIG. 7 is a flow diagram illustrating an example technique for delivering electrical stimulation according to a specific pattern of pulses having different pulse frequencies. For convenience, FIG. 7 is described with respect to IMD 200 of FIG. 2. However, the techniques of FIG. 7 may be performed by different components of IMD 200 or by additional or alternative medical devices, such as programmer 300.

In the example of FIG. 7, processing circuitry 210 determines the pattern of the first and second stimulation to be delivered to the patient (700). This pattern may include determining which slots of a series of slots includes respective pulses for prime stimulation and base stimulation or otherwise determining the manner in which pulses of a first stimulation delivered to a first target tissue (e.g., glial cells) will be interleaved with pulses of a second stimulation delivered to a second target tissue (e.g., neurons). Processing circuitry 210 then delivers the first stimulation interleaved with the second stimulation (702) until cycling instructions indicate to turn off stimulation (704). If instructions indicate that the time period for stimulation has not elapsed (“NO” branch of block 704), processing circuitry 210 continues to deliver stimulation. If instructions indicate that the time period for stimulation has elapsed (“YES” branch of block 704), processing circuitry 210 withholds or ceases stimulation delivery (706). In response to the time period of no stimulation elapsing (“YES” branch of block 708), processing circuitry 210 again delivers stimulation (702).

In one example, the stimulation on time for prime pulses may be 15 minutes while the stimulation off time for prime pulses may be 30 minutes, which results in an on time to off time ratio of 1:2. The base pulses may be cycled at a different durations and/or different times. Example on durations may be as short as 15 seconds or as long as 30 minutes. Off durations may be within a range of 15 seconds to as long as 60 minutes. In other examples, the on and off periods of each cycle may be longer or shorter than these ranges. However, these are only examples, as the prime pulses may be delivered for shorter or greater durations before cycling off, and remaining off for shorter or longer times. In addition, the duty cycle of the on and off cycling ratio may be less or greater than the 1:2 ratio of the above example.

In some examples, processing circuitry 210 may increase the stimulation on time if therapy is not effective and/or decrease the stimulation on time if therapy is effective in order to find a balance between effective therapy and reduced power consumption. In some examples, the instructions that cause processing circuitry 210 to cycle stimulation off or cycle stimulation back on may be based on one or more of therapy efficacy as determined by user input indicating therapy is effective, sensed or manual input indicative of patient pain levels, detected patient activity levels that indicate therapy enables the patient to perform certain levels of activity, or sensed ECAP signals (e.g., ECAP amplitudes that may indicate nerve signal propagation is suppressed by the prime pulses). In this manner, cycling stimulation on and off may be determined by time schedules, automatically sensed data, and/or user input. In one example, processing circuitry 210 may cycle prime pulses on in response to activity of the patient, such as in response to the patient assuming certain posture states (e.g., the patient has more pain when lying down) or starting certain activities (e.g., walking may create more pain for the patient).

In other examples, processing circuitry 210 may cycle the base pulses and/or the prime pulses based on the selected frequencies for each type of pulse. For example, higher frequency prime pulses may have reduced on times for the cycle (in absolute time and/or percentage of the total duty cycle) when compared to lower frequency prime pulses.

In some examples, the process of cycling stimulation on and off in FIG. 7 may be adjusted such that the prime pulses are cycled separately from the base pulses being cycled. For example, the prime pulse may be cycled on and off at a different frequency and/or a different on and/or off times than the frequency and on and/or off times of the base pulses. Or, in one example, processing circuitry 210 may cycle the prime pulses on and off while continually delivering the base pulses. In some examples, the prime pulses may be delivered to prime glial cells, which may operate on a different timeline than neurons affected by the base pulses. Glial cells may operate on the order of minutes such that IMD 200 can cycle prime pulses on for 5-10 minutes in order to achieve the benefit of glial cell stimulation and then cycle prime pulses off for 5-10 minutes. IMD 200 may deliver base pulses continuously as prime pulses are cycled on and off, or base pulses may be delivered during the off cycle for the prime pulses.

In some examples, processing circuitry 210 may cycle (or shift) between different electrode combinations for the prime pulses and/or the base pulses. This shift between electrode combinations can be done instead of turning stimulation on or off, or the shift can happen during the stimulation on time to preserve the off portion of the cycle. For example, processing circuitry 210 may move the prime pulses to different electrode combinations at a predetermined frequency or in response to various trigger events such as above. In this manner, processing circuitry 210 may enable the system to target different anatomical locations, such as different glial cells, over time while reducing the overall consumption of energy if those locations were to receive stimulation at all times.

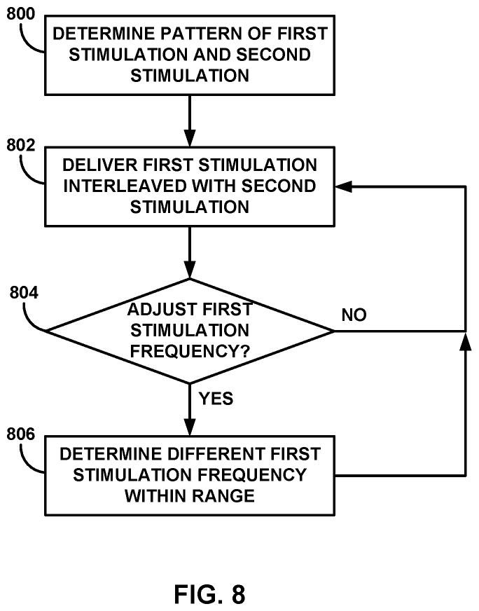

FIG. 8 is a flow diagram illustrating an example technique for adjusting the frequency of prime stimulation pulses within range of frequencies. For convenience, FIG. 8 is described with respect to IMD 200 of FIG. 2. However, the techniques of FIG. 8 may be performed by different components of IMD 200 or by additional or alternative medical devices, such as programmer 300.

In the example of FIG. 8, processing circuitry 210 determines the pattern of the first and second stimulation to be delivered to the patient (800). This pattern may include determining which slots of a series of slots includes respective pulses for prime stimulation and base stimulation or otherwise determining the manner in which pulses of a first stimulation delivered to a first target tissue (e.g., glial cells) will be interleaved with pulses of a second stimulation delivered to a second target tissue (e.g., neurons). Processing circuitry 210 then determines whether to adjust the pulse frequency of the first stimulation (804). In some examples, processing circuitry 210 includes instructions for adjusting the prime stimulation pulse frequency according to a certain schedule or at predetermined intervals. Each adjustment may be within a predetermined range of frequencies or varied randomly or pseudo randomly such that the pulse frequency is varied while staying within the predetermined range. In some examples, processing circuitry 210 includes a random pulse generator or operate using a stochastic function in order to generate pulses that fluctuate with random pulse frequencies (or random inter-pulse intervals). Processing circuitry 210 may randomize the pulse frequency around a center target pulse frequency and within the range of pulse frequencies. The range may be a percentage of the target frequency, centered above and below the target frequency. For example, the range may be equal to a frequency in the range of 20 percent to 100 percent of the target frequency. An example average frequency for the target pulse frequency may fall within any ranges herein, such as a pulse frequency at or above 200 Hz when the pulse frequency is averaged over a period of time, such as 1 second, 10 seconds, 30 seconds, 1 minute, 10 minutes, or any other predetermined period of time. In some examples, adjustment of the pulse frequency may include selecting a different pattern of pulses as described with respect to FIGS. 4, 5, and 6. In some examples, processing circuitry 210 may be configured to also, or instead, randomize the pulse frequency of the second stimulation pulses (e.g., the base pulses) about a target frequency and/or within a respective frequency range.

If instructions indicate that processing circuitry 210 should not adjust the frequency of the first stimulation, (“NO” branch of block 804), processing circuitry 210 continues to deliver stimulation (802). If instructions indicate that processing circuitry 210 should adjust the frequency of the first stimulation, (“YES” branch of block 804), processing circuitry 210 determines the different stimulation frequency within the range and changes the pulse frequency to that new different stimulation frequency (806). Adjusting the frequency within the range of frequencies may maintain efficacy of the stimulation while reducing accommodation to the stimulation and/or reducing long term energy consumption by using lower frequencies when possible. Processing circuitry 210 then continues to deliver stimulation (802). Instead of describing the variation as a variation to frequency, processing circuitry 210 may effectively adjust the frequency by varying the interpulse interval between pulses in a similar fashion (e.g., within a range around a target interpulse interval).

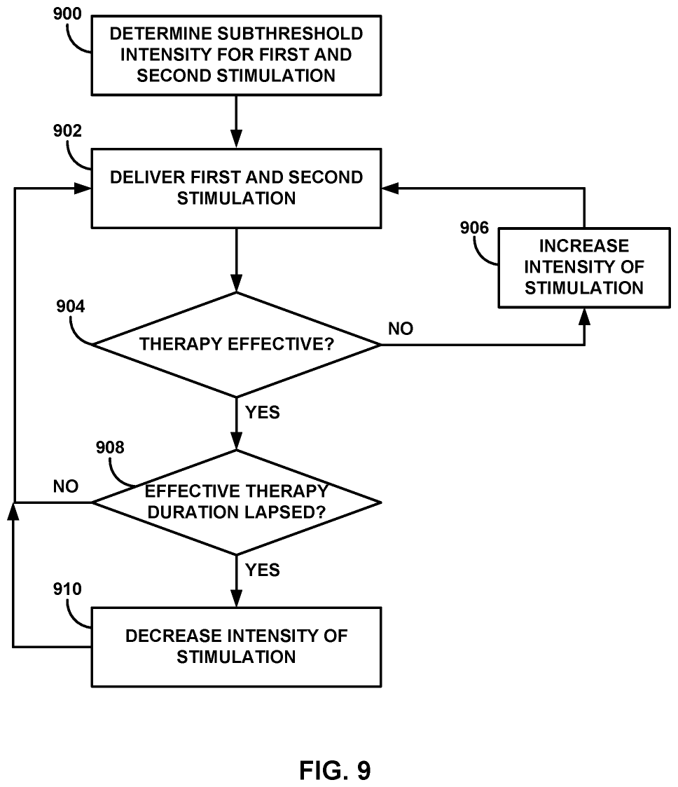

FIG. 9 is a flow diagram illustrating an example technique for reducing stimulation intensity of first stimulation and/or second stimulation over time while maintaining effective therapy. For convenience, FIG. 9 is described with respect to IMD 200 of FIG. 2. However, the techniques of FIG. 9 may be performed by different components of IMD 200 or by additional or alternative medical devices, such as programmer 300.

In the example of FIG. 9, processing circuitry 210 determines the subthreshold intensity for first and second stimulation (e.g., prime and base stimulation pulses) (900). This process may include determining the perception threshold or sensory threshold for the patient for one or both of the first and second stimulation. In some examples, the threshold may depend on the frequency of pulses delivered, so the system may determine the threshold intensity for the prime and/or base pulses after the pulse frequencies have been selected. These thresholds may be the same or different for the first and second stimulation. Processing circuitry 210 may then reduce the intensity for the first and second stimulation by some percentage or some absolute amplitude. In one example, processing circuitry 210 may determine the subthreshold intensity by calculating a percentage of the threshold intensity (e.g., 40 percent, 50 percent, 60 percent, 70 percent, etc.). An example range of initial threshold intensities may be from 40 percent to 70 percent of the threshold intensity. In one example, processing circuitry 210 may start at percent of the threshold intensity for prime pulses. Some examples may start at intensities less than 40 percent of the threshold intensity for the prime pulses and/or the base pulses. In some examples, processing circuitry 210 may perform the process of FIG. 9 for the base pulses first (e.g., ramping up intensity from the sub-threshold intensity until efficacy is identified) and then ramp up the intensity for the prime pulses until efficacy is reached. The initial sub-threshold percentages may be different for the first and second stimulation pulses. The intensity may refer to current or voltage amplitude, pulse width, frequency, or some combination thereof. In some examples, the intensity may refer to the charge density of a pulse.

Processing circuitry 210 then delivers the first and second stimulation to the patient (902). If processing circuitry 210 receives input that the therapy is not effective (“NO” branch of block 904), processing circuitry 210 increases the intensity of the first and/or second stimulation pulses (906) before again delivering the first and second stimulation (902). Each step of intensity increase may be predetermined as a set amplitude step or percentage step. For example, the increase step may be selected from a range such as 0.1 mA to 1 mA current amplitude or 1 percent to 10 percent of the threshold intensity. In one example, each intensity increase may be 0.2 mA current amplitude. In another example, each intensity increase may be 2 percent of the threshold intensity. If processing circuitry 210 receives input that the therapy is effective (“YES” branch of block 908), processing circuitry 210 determines if the effective therapy duration has lapsed which is a period of time during which effective therapy has been delivered (908). This effective therapy duration may be predetermined and indicative of therapy that provides relief to the patient may be reduced to conserve power while maintaining efficacy. This duration may be on the order of hours, days, weeks, or months. If the effective therapy duration has not lapsed (“NO” branch of block 908), processing circuitry 210 continues to deliver first and second stimulation (902). If the effective therapy duration has lapsed (“YES” branch of block 908), processing circuitry 210 decreases the intensity of the first and/or second stimulation pulses (910) for delivery of stimulation (902). The reduction in stimulation intensity may retain therapy efficacy while reducing power consumption for some period of time. The process of FIG. 9 may continue to identify the lowest intensity of stimulation that can maintain effective therapy.

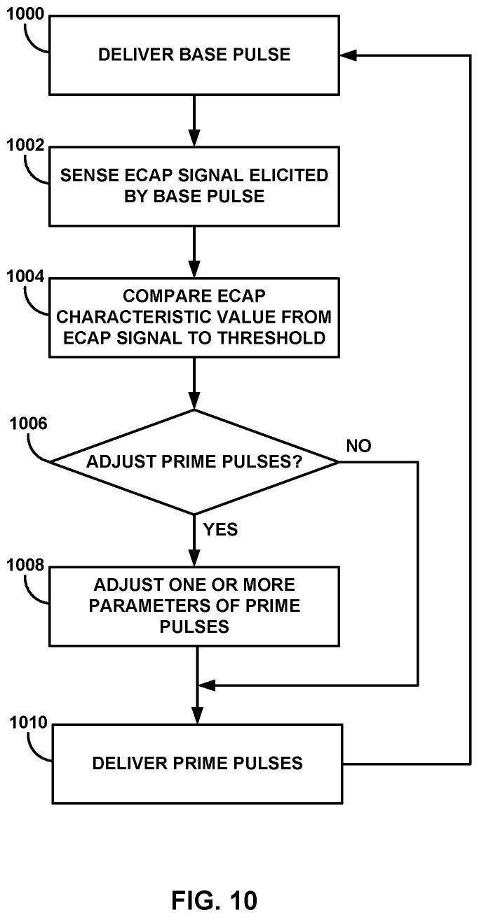

FIG. 10 is a flow diagram illustrating an example technique for adjusting a parameter value that defines prime stimulation pulses based on an evoked compound action potential (ECAP) elicited by a base stimulation pulse. For convenience, FIG. 10 is described with respect to IMD 200 of FIG. 2. However, the techniques of FIG. 10 may be performed by different components of IMD 200 or by additional or alternative medical devices, such as programmer 300.

In the example of FIG. 10, processing circuitry 210 controls IMD 200 to deliver base pulses and prime pulses as described herein. IMD 200 may use ECAP signals elicited by respective base pulses to modulate one or more parameters that define subsequent prime pulses. The prime pulses may not elicit a detectable ECAP or be delivered at too high a frequency to detect ECAP signals off of a prime pulse. In some examples, the prime pulses may be delivered at a sub-perception threshold intensity such that ECAPs are not elicited, but base pulses may be delivered with an intensity that elicits an ECAP. However, in other examples, ECAPs may be elicited and detected for use as feedback for adjusting prime pulses.

Processing circuitry 210 controls IMD 200 to deliver a base pulse as part of the overall therapy (1000). Then, processing circuitry 210 can control IMD 200 to sense an ECAP signal elicited by the delivered base pulse (1002). In some examples, the base pulse amplitude may be increased to be above a threshold perception level (or threshold ECAP detection level) for the base pulse when IMD 200 is scheduled to detect an ECAP in response to delivery of that base pulse. Other base pulses after which an ECAP is not scheduled to be detected may be set to a sub-threshold intensity or amplitude as appropriate for therapy. In other words, processing circuitry 210 may change the intensity of a base pulse from therapeutic levels if necessary to elicit a detectable ECAP. Processing circuitry 210 may compare an ECAP characteristic value from the ECAP signal to a threshold. The ECAP characteristic value may be an amplitude of one or more peaks within the ECAP signal, an amplitude between adjacent positive and negative peaks of the ECAP signal, an area under one or more curves of the ECAP signal, or any other characteristic representative of the number of nerves activated by the base pulse. The threshold may be an upper threshold indicative of a pain or discomfort threshold of the patient, or some value that triggers changing one or more parameter values defining the prime pulses.

If processing circuitry 210 determines that the ECAP characteristic value does not exceed the threshold, for example, (“NO” branch of block 1006), processing circuitry 210 continues to control IMD 200 to deliver subsequent prime pulses (1010). If processing circuitry 210 determines that the ECAP characteristic value does exceed the threshold, for example, (“YES” branch of block 1006), processing circuitry 210 adjusts one or more parameters that defines subsequent prime pulses (1008). For example, processing circuitry 210 may reduce the current amplitude of subsequent prime pulses until the ECAP characteristic value no longer exceeds the threshold. Processing circuitry 210 may additionally or alternatively adjust a pulse frequency, pulse width, duty cycle or cycling duration, or any other parameter in order to bring the ECAP characteristic value back below, above, or to the threshold. Processing circuitry 210 then controls IMD 200 to continue to deliver the prime pulses (1010) and the next base pulse (1000). In some examples, processing circuitry 210 does not sense ECAP signals or determine ECAP characteristic values for each base pulses delivered. In some examples, processing circuitry 210 may reduce the “on” time of prime and/or base pulses when cycling the stimulation on and off in addition or as an alternative to decreasing intensity of stimulation.

The following examples are described herein. Example 1: A method includes generating, by stimulation generation circuitry, a first train of electrical stimulation pulses at a first frequency to a first target tissue; and generating, by the stimulation generation circuitry, a second train of electrical stimulation pulses at a second frequency to a second target tissue different from the first target tissue, wherein at least some electrical stimulation pulses of the first train of electrical stimulation pulses are interleaved with at least some electrical stimulation pulses of the second train of electrical stimulation pulses, and wherein the first frequency is greater than the second frequency.

Example 2: The method of example 1, further comprising generating, by stimulation generation circuitry, a third train of electrical stimulation pulses at a third frequency to the first target tissue, wherein at least some electrical stimulation pulses of the first train of electrical stimulation pulses, at least some of the electrical stimulation pulses of the second train of electrical stimulation pulses, and at least some of the electrical stimulation pulses of the third train of electrical stimulation pulses are all interleaved together.

Example 3: The method of example 2, further comprising changing an order of pulses of the first train of electrical stimulation pulses with pulses of the third train of electrical stimulation pulses over time to adjust a pulse pattern created by interleaving the at least some of the electrical stimulation pulses of the first train of electrical stimulation pulses with the at least some of the electrical stimulation pulses of the third train of electrical stimulation pulses.

Example 4: The method of any of examples 2 and 3, wherein the first train of electrical stimulation pulses and the third train of electrical stimulation pulses are generated together with an average frequency greater than the second frequency of the second train of electrical stimulation pulses.

Example 5: The method of example 4, wherein the third frequency one of equal to the second frequency or greater than the second frequency.

Example 6: The method of any of examples 3 through 5, wherein the average frequency is selected from a frequency range from approximately 150 Hz to approximately 600 Hz.

Example 7: The method of any of examples 1 through 6, wherein the stimulation generation circuitry is configured to generate electrical stimulation pulses in a repeatable series of slots, the repeatable series of slots being repeatable over time for generating the first train of electrical stimulation pulses and the second train of electrical stimulation pulses, and wherein: generating the first train of electrical stimulation pulses comprises generating one pulse for a first slot of at least some of the repeatable series of slots that achieves the first frequency, and generating the second train of electrical stimulation pulses comprises generating one pulse for a second slot of at least some of the repeatable series of slots that achieves the second frequency.

Example 8: The method of any of examples 1 through 7, wherein the second frequency is selected from a frequency range from approximately 40 Hz to approximately 60 Hz.

Example 9: The method of any of examples 1 through 8, further comprising cycling between a first mode of a first period of time and a second mode of a second period of time, wherein the first mode comprises generating the first train of electrical stimulation pulses at least partially interleaved with the second train of electrical stimulation pulses, and wherein the second mode comprises withholding generation of the first train of electrical stimulation pulses and the second train of electrical stimulation pulses.

Example 10: The method of example 9, wherein a ratio of the first period to the second period of time is selected in a range from approximately 1:1 to 1:3.

Example 11: The method of any of examples 9 and 10, wherein the first period of time is selected from a range from approximately 1 minute to approximately 30 minutes.

Example 12: The method of any of examples 9 through 11, wherein the first period of time is selected from a range from approximately 5 minute to approximately 15 minutes.

Example 13: The method of any of examples 1 through 12, wherein an amplitude of pulses of the first train of electrical stimulation pulses is below at least one of a perception threshold or a sensory threshold of a patient.

Example 14: The method of any of examples 1 through 13, wherein an amplitude of pulses of the second train of electrical stimulation pulses is below at least one of a perception threshold or a sensory threshold of a patient.

Example 15: The method of any of examples 1 through 14, further includes ceasing generating the first train of electrical stimulation pulses at the first frequency to the first target tissue; and generating the first train of electrical stimulation pulses at the first frequency to a third target tissue different from the first target tissue.

Example 16: The method of any of examples 1 through 15, wherein the first target tissue comprises glial cells, and wherein the second target tissue comprises neurons.

Example 17: A system that includes stimulation generation circuitry configured to generate and deliver electrical stimulation therapy; and processing circuitry configured to control the stimulation generation circuitry to: generate a first train of electrical stimulation pulses at a first frequency to a first target tissue; and generate a second train of electrical stimulation pulses at a second frequency to a second target tissue different from the first target tissue, wherein at least some electrical stimulation pulses of the first train of electrical stimulation pulses are interleaved with at least some electrical stimulation pulses of the second train of electrical stimulation pulses, and wherein the first frequency is greater than the second frequency.

Example 18: The system of example 17, wherein the processing circuitry is configured to control the stimulation generation circuitry to generate a third train of electrical stimulation pulses at a third frequency to the first target tissue, wherein at least some electrical stimulation pulses of the first train of electrical stimulation pulses, at least some of the electrical stimulation pulses of the second train of electrical stimulation pulses, and at least some of the electrical stimulation pulses of the third train of electrical stimulation pulses are all interleaved together.

Example 19: The system of example 18, wherein the processing circuitry is configured to control the stimulation generation circuitry to change an order of pulses of the first train of electrical stimulation pulses with pulses of the third train of electrical stimulation pulses over time to adjust a pulse pattern created by interleaving the at least some of the electrical stimulation pulses of the first train of electrical stimulation pulses with the at least some of the electrical stimulation pulses of the third train of electrical stimulation pulses.

Example 20: The system of example 18, wherein the first train of electrical stimulation pulses and the third train of electrical stimulation pulses are generated together with an average frequency greater than the second frequency of the second train of electrical stimulation pulses.

Example 21: The system of example 20, wherein the third frequency one of equal to the second frequency or greater than the second frequency.

Example 22: The system of any of examples 19 and 20, wherein the average frequency is selected from a frequency range from approximately 150 Hz to approximately 600 Hz.

Example 23: The system of any of examples 17 through 21, wherein the stimulation generation circuitry is configured to generate electrical stimulation pulses in a repeatable series of slots, the repeatable series of slots being repeatable over time for generating the first train of electrical stimulation pulses and the second train of electrical stimulation pulses, and wherein: the processing circuitry is configured to control the stimulation generation circuitry to generate the first train of electrical stimulation pulses by at least generating one pulse for a first slot of at least some of the repeatable series of slots that achieves the first frequency, and the processing circuitry is configured to control the stimulation generation circuitry to generate the second train of electrical stimulation pulses by at least generating one pulse for a second slot of at least some of the repeatable series of slots that achieves the second frequency.

Example 24: The system of any of examples 17 through 22, wherein the second frequency is selected from a frequency range from approximately 40 Hz to approximately 60 Hz.

Example 25: The system of any of examples 17 through 23, wherein the processing circuitry is configured to control the stimulation generation circuitry to cycle between a first mode of a first period of time and a second mode of a second period of time, wherein the first mode comprises generating the first train of electrical stimulation pulses at least partially interleaved with the second train of electrical stimulation pulses, and wherein the second mode comprises withholding generation of the first train of electrical stimulation pulses and the second train of electrical stimulation pulses.

Example 26: The system of example 25, wherein a ratio of the first period to the second period of time is selected in a range from approximately 1:1 to 1:3.

Example 27: The system of example 25, wherein the first period of time is selected from a range from approximately 1 minute to approximately 30 minutes.

Example 28: The system of any of examples 25 and 26, wherein the first period of time is selected from a range from approximately 5 minute to approximately 15 minutes.

Example 29: The system of any of examples 17 through 27, wherein an amplitude of pulses of the first train of electrical stimulation pulses is below at least one of a perception threshold or a sensory threshold of a patient.

Example 30: The system of any of examples 17 through 28, wherein an amplitude of pulses of the second train of electrical stimulation pulses is below at least one of a perception threshold or a sensory threshold of a patient.

Example 31: The system of any of examples 17 through 29, the processing circuitry is configured to control the stimulation generation circuitry to: cease generating the first train of electrical stimulation pulses at the first frequency to the first target tissue; and generate the first train of electrical stimulation pulses at the first frequency to a third target tissue different from the first target tissue.

Example 32: The system of any of examples 17 through 30, wherein the first target tissue comprises glial cells, and wherein the second target tissue comprises neurons.

Example 33: The system of any of examples 17 through 31, further comprising an implantable medical device comprising the processing circuitry and the stimulation generation circuitry.

Example 34: A non-transitory computer-readable medium that includes instructions that, when executed, cause processing circuitry to: control stimulation generation circuitry to: generate a first train of electrical stimulation pulses at a first frequency to a first target tissue; and generate a second train of electrical stimulation pulses at a second frequency to a second target tissue different from the first target tissue, wherein at least some electrical stimulation pulses of the first train of electrical stimulation pulses are interleaved with at least some electrical stimulation pulses of the second train of electrical stimulation pulses, and wherein the first frequency is greater than the second frequency.

Example 35: An implantable medical device configured to perform the method of any of examples 1 through 16.

Example 36: An external programming device configured to program a medical device to perform the method of any of examples 1 through 16.

Example 37: A system comprising stimulation means for performing the method of any of examples 1 through 16.

Example 38: A method comprising any combination of the methods of examples 1 through 16.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, FRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1. A system comprising:
stimulation generation circuitry configured to generate and deliver electrical stimulation therapy; and
processing circuitry configured to control the stimulation generation circuitry to:
generate a first train of electrical stimulation pulses at a first frequency to a first target tissue; and
generate a second train of electrical stimulation pulses at a second frequency to a second target tissue different from the first target tissue, wherein at least some electrical stimulation pulses of the first train of electrical stimulation pulses are interleaved with at least some electrical stimulation pulses of the second train of electrical stimulation pulses, and wherein the first frequency is greater than the second frequency.

2. The system of claim 1, wherein the processing circuitry is configured to control the stimulation generation circuitry to generate a third train of electrical stimulation pulses at a third frequency to the first target tissue, wherein at least some electrical stimulation pulses of the first train of electrical stimulation pulses, at least some of the electrical stimulation pulses of the second train of electrical stimulation pulses, and at least some of the electrical stimulation pulses of the third train of electrical stimulation pulses are all interleaved together.

3. The system of claim 2, wherein the processing circuitry is configured to control the stimulation generation circuitry to change an order of pulses of the first train of electrical stimulation pulses with pulses of the third train of electrical stimulation pulses over time to adjust a pulse pattern created by interleaving the at least some of the electrical stimulation pulses of the first train of electrical stimulation pulses with the at least some of the electrical stimulation pulses of the third train of electrical stimulation pulses.

4. The system of claim 2, wherein the first train of electrical stimulation pulses and the third train of electrical stimulation pulses are generated together with an average frequency greater than the second frequency of the second train of electrical stimulation pulses.

5. The system of claim 4, wherein the third frequency one of equal to the second frequency or greater than the second frequency.

6. The system of claim 3, wherein the average frequency is selected from a frequency range from approximately 150 Hz to approximately 600 Hz.

7. The system of claim 1, wherein the stimulation generation circuitry is configured to generate electrical stimulation pulses in a repeatable series of slots, the repeatable series of slots being repeatable over time for generating the first train of electrical stimulation pulses and the second train of electrical stimulation pulses, and wherein:
the processing circuitry is configured to control the stimulation generation circuitry to generate the first train of electrical stimulation pulses by at least generating one pulse for a first slot of at least some of the repeatable series of slots that achieves the first frequency, and
the processing circuitry is configured to control the stimulation generation circuitry to generate the second train of electrical stimulation pulses by at least generating one pulse for a second slot of at least some of the repeatable series of slots that achieves the second frequency.

8. The system of claim 1, wherein the second frequency is selected from a frequency range from approximately 40 Hz to approximately 60 Hz.

9. The system of claim 1, wherein the processing circuitry is configured to control the stimulation generation circuitry to cycle between a first mode of a first period of time and a second mode of a second period of time, wherein the first mode comprises generating the first train of electrical stimulation pulses at least partially interleaved with the second train of electrical stimulation pulses, and wherein the second mode comprises withholding generation of the first train of electrical stimulation pulses and the second train of electrical stimulation pulses.

10. The system of claim 9, wherein a ratio of the first period to the second period of time is selected in a range from approximately 1:1 to 1:3.

11. The system of claim 9, wherein the first period of time is selected from a range from approximately 1 minute to approximately 30 minutes.

12. The system of claim 9, wherein the first period of time is selected from a range from approximately 5 minute to approximately 15 minutes.

13. The system of claim 1, wherein an amplitude of at least one of pulses of the first train of electrical stimulation pulses or pulses of the second train of electrical stimulation is below at least one of a perception threshold or a sensory threshold of a patient.

14. The system of claim 1, wherein the first target tissue comprises glial cells, and wherein the second target tissue comprises neurons.

15. The system of claim 1, further comprising an implantable medical device comprising the processing circuitry and the stimulation generation circuitry.

16. A method comprising:
generating, by stimulation generation circuitry, a first train of electrical stimulation pulses at a first frequency to a first target tissue; and
generating, by the stimulation generation circuitry, a second train of electrical stimulation pulses at a second frequency to a second target tissue different from the first target tissue, wherein at least some electrical stimulation pulses of the first train of electrical stimulation pulses are interleaved with at least some electrical stimulation pulses of the second train of electrical stimulation pulses, and wherein the first frequency is greater than the second frequency.

17. The method of claim 16, further comprising generating, by stimulation generation circuitry, a third train of electrical stimulation pulses at a third frequency to the first target tissue, wherein at least some electrical stimulation pulses of the first train of electrical stimulation pulses, at least some of the electrical stimulation pulses of the second train of electrical stimulation pulses, and at least some of the electrical stimulation pulses of the third train of electrical stimulation pulses are all interleaved together.

18. The method of claim 17, further comprising changing an order of pulses of the first train of electrical stimulation pulses with pulses of the third train of electrical stimulation pulses over time to adjust a pulse pattern created by interleaving the at least some of the electrical stimulation pulses of the first train of electrical stimulation pulses with the at least some of the electrical stimulation pulses of the third train of electrical stimulation pulses, wherein:
the first train of electrical stimulation pulses and the third train of electrical stimulation pulses are generated together with an average frequency greater than the second frequency of the second train of electrical stimulation pulses,
the third frequency one of equal to the second frequency or greater than the second frequency, and
the average frequency is selected from a frequency range from approximately 150 Hz to approximately 600 Hz.

19. The method of claim 16, further comprising cycling between a first mode of a first period of time and a second mode of a second period of time, wherein the first mode comprises generating the first train of electrical stimulation pulses at least partially interleaved with the second train of electrical stimulation pulses, and wherein the second mode comprises withholding generation of the first train of electrical stimulation pulses and the second train of electrical stimulation pulses.

20. A non-transitory computer-readable medium that comprises instructions that, when executed, cause processing circuitry to:
control stimulation generation circuitry to:
generate a first train of electrical stimulation pulses at a first frequency to a first target tissue; and
generate a second train of electrical stimulation pulses at a second frequency to a second target tissue different from the first target tissue, wherein at least some electrical stimulation pulses of the first train of electrical stimulation pulses are interleaved with at least some electrical stimulation pulses of the second train of electrical stimulation pulses, and wherein the first frequency is greater than the second frequency.
